SEQUENCE LISTING (25 sequences)

SEQ ID NO: 1 - DNA, 5139 bp, Mus musculus
  source 1..5139: mol_type = genomic DNA
agagaggagagccatataaagagccagcggctacagccccagctcgcctctgcccacccctgccccttaccccttcattccttccacctttttccttcactatgggaccagcttcagggtcccagctactagtgctactgctgctgttggccagctccccattagctctggggatccccatgtattccatcattactcccaatgtcctacggctggagagcgaagagaccatcgtactggaggcccacgatgctcagggtgacatcccagtcacagtcactgtgcaagacttcctaaagaggcaagtgctgaccagtgagaagacagtgttgacaggagccagtggacatctgagaagcgtctccatcaagattccagccagtaaggaattcaactcagataaggaggggcacaagtacgtgacagtggtggcaaacttcggggaaacggtggtggagaaagcagtgatggtaagcttccagagtgggtacctcttcatccagacagacaagaccatctacacccctggctccactgtcttatatcggatcttcactgtggacaacaacctactgcccgtgggcaagacagtcgtcatcctcattgagacccccgatggcattcctgtcaagagagacattctgtcttccaacaaccaacacggcatcttgcctttgtcttggaacattcctgaactggtcaacatggggcagtggaagatccgagccttttacgaacatgcgccgaagcagatcttctccgcagagtttgaggtgaaggaatacgtgctgcccagttttgaggtccgggtggagcccacagagacattttattacatcgatgacccaaatggcctggaagtttccatcatagccaagttcctgtacgggaaaaacgtggacgggacagccttcgtgatttttggggtccaggatggcgataagaagatttctctggcccactccctcacgcgcgtagtgattgaggatggtgtgggggatgcagtgctgacccggaaggtgctgatggagggggtacggccttccaacgccgacgccctggtggggaagtccctgtatgtctccgtcactgtcatcctgcactcaggtagtgacatggtagaggcagagcgcagtgggatcccgattgtcacttccccgtaccagatccacttcaccaagacacccaaattcttcaagccagccatgccctttgacctcatggtgttcgtgaccaaccccgatggctctccggccagcaaagtgctggtggtcactcagggatctaatgcaaaggctctcacccaagatgatggcgtggccaagctaagcatcaacacacccaacagccgccaacccctgaccatcacagtccgcaccaagaaggacactctcccagaatcacggcaggccaccaagacaatggaggcccatccctacagcactatgcacaactccaacaactacctacacttgtcagtgtcacgaatggagctcaagccgggggacaacctcaatgtcaacttccacctgcgcacagacccaggccatgaggccaagatccgatactacacctacctggttatgaacaaggggaagctcctgaaggcaggccgccaggttcgggagcctggccaggacctggtggtcttgtccctgcccatcactccagagtttattccttcatttcgcctggtggcttactacaccctgattggagctagtggccagagggaggtggtggctgactctgtgtgggtggatgtgaaggattcctgtattggcacgctggtggtgaagggtgacccaagagataaccatctcgcacctgggcaacaaacgacactcaggattgaaggaaaccagggggcccgagtggggctagtggctgtggacaagggagtgtttgtgctgaacaagaagaacaaactcacacagagcaagatctgggatgtggtagagaaggcagacattggctgcaccccaggcagtgggaagaactatgctggtgtcttcatggatgcaggcctggccttcaagacaagccaaggactgcagactgaacagagagcagatcttgagtgcaccaagccagcagcccgccgccgtcgctcagtacagttgatggaaagaaggatggacaaagctggtcagtacactgacaagggtcttcggaagtgttgtgaggatggtatgcgggatatccctatgagatacagctgccagcgccgggcacgcctcatcacccagggcgagaactgcataaaggccttcatagactgctgcaaccacatcaccaagctgcgtgaacaacacagaagagaccacgtgctgggcctggccaggagtgaattggaggaagacataattccagaagaagatattatctctagaagccacttcccacagagctggttgtggaccatagaagagttgaaagaaccagagaaaaatggaatctctacgaaggtcatgaacatctttctcaaagattccatcaccacctgggagattctggcagtgagcttgtcagacaagaaagggatctgtgtggcagacccctatgagatcagagtgatgcaggacttcttcattgacctgcggctgccctactctgtagtgcgcaacgaacaggtggagatcagagctgtgctcttcaactaccgtgaacaggaggaacttaaggtgagggtggaactgttgcataatccagccttctgcagcatggccaccgccaagaatcgctacttccagaccatcaaaatccctcccaagtcctcggtggctgtaccgtatgtcattgtccccttgaagatcggccaacaagaggtggaggtcaaggctgctgtcttcaatcacttcatcagtgatggtgtcaagaagacactgaaggtcgtgccagaaggaatgagaatcaacaaaactgtggccatccatacactggacccagagaagctcggtcaagggggagtgcagaaggtggatgtgcctgccgcagaccttagcgaccaagtgccagacacagactctgagaccagaattatcctgcaagggagcccggtggttcagatggctgaagatgctgtggacggggagcggctgaaacacctgatcgtgacccccgcaggctgtggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagagaccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagccgtagctctgcaaccacgtttcgcctgctctgggaaaatggcaacctcctgcgatcggaagagaccaagcaaaatgaggccttctctctaacagccaaaggaaaaggccgaggcacattgtcggtggtggcagtgtatcatgccaaactcaaaagcaaagtcacctgcaagaagtttgacctcagggtcagcataagaccagcccctgagacagccaagaagcccgaggaagccaagaataccatgttccttgaaatctgcaccaagtacttgggagatgtggacgccactatgtccatcctggacatctccatgatgactggctttgctccagacacaaaggacctggaactgctggcctctggagtagatagatacatctccaagtacgagatgaacaaagccttctccaacaagaacaccctcatcatctacctagaaaagatttcacacaccgaagaagactgcctgaccttcaaagttcaccagtactttaatgtgggacttatccagcccgggtcggtcaaggtctactcctattacaacctcgaggaatcatgcacccggttctatcatccagagaaggacgatgggatgctcagcaagctgtgccacagtgaaatgtgccggtgtgctgaagagaactgcttcatgcaacagtcacaggagaagatcaacctgaatgtccggctagacaaggcttgtgagcccggagtcgactatgtgtacaagaccgagctaaccaacatagagctgttggatgattttgatgagtacaccatgaccatccagcaggtcatcaagtcaggctcagatgaggtgcaggcagggcagcaacgcaagttcatcagccacatcaagtgcagaaacgccctgaagctgcagaaagggaagaagtacctcatgtggggcctctcctctgacctctggggagaaaagcccaacaccagctacatcattgggaaggacacgtgggtggagcactggcctgaggcagaagaatgccaggatcagaagtaccagaaacagtgcgaagaacttggggcattcacagaatctatggtggtttatggttgtcccaactgactacagcccagccctctaataaagcttcagttgtatttcacccatc

SEQ ID NO: 2 - protein, 1663 aa, Mus musculus
MGPASGSQLLVLLLLLASSPLALGIPMYSIITPNVLRLESEETIVLEAHDAQGDIPVTVTVQDFLKRQVLTSEKTVLTGASGHLRSVSIKIPASKEFNSDKEGHKYVTVVANFGETVVEKAVMVSFQSGYLFIQTDKTIYTPGSTVLYRIFTVDNNLLPVGKTVVILIETPDGIPVKRDILSSNNQHGILPLSWNIPELVNMGQWKIRAFYEHAPKQIFSAEFEVKEYVLPSFEVRVEPTETFYYIDDPNGLEVSIIAKFLYGKNVDGTAFVIFGVQDGDKKISLAHSLTRVVIEDGVGDAVLTRKVLMEGVRPSNADALVGKSLYVSVTVILHSGSDMVEAERSGIPIVTSPYQIHFTKTPKFFKPAMPFDLMVFVTNPDGSPASKVLVVTQGSNAKALTQDDGVAKLSINTPNSRQPLTITVRTKKDTLPESRQATKTMEAHPYSTMHNSNNYLHLSVSRMELKPGDNLNVNFHLRTDPGHEAKIRYYTYLVMNKGKLLKAGRQVREPGQDLVVLSLPITPEFIPSFRLVAYYTLIGASGQREVVADSVWVDVKDSCIGTLVVKGDPRDNHLAPGQQTTLRIEGNQGARVGLVAVDKGVFVLNKKNKLTQSKIWDVVEKADIGCTPGSGKNYAGVFMDAGLAFKTSQGLQTEQRADLECTKPAARRRRSVQLMERRMDKAGQYTDKGLRKCCEDGMRDIPMRYSCQRRARLITQGENCIKAFIDCCNHITKLREQHRRDHVLGLARSELEEDIIPEEDIISRSHFPQSWLWTIEELKEPEKNGISTKVMNIFLKDSITTWEILAVSLSDKKGICVADPYEIRVMQDFFIDLRLPYSVVRNEQVEIRAVLFNYREQEELKVRVELLHNPAFCSMATAKNRYFQTIKIPPKSSVAVPYVIVPLKIGQQEVEVKAAVFNHFISDGVKKTLKVVPEGMRINKTVAIHTLDPEKLGQGGVQKVDVPAADLSDQVPDTDSETRIILQGSPVVQMAEDAVDGERLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRSSATTFRLLWENGNLLRSEETKQNEAFSLTAKGKGRGTLSVVAVYHAKLKSKVTCKKFDLRVSIRPAPETAKKPEEAKNTMFLEICTKYLGDVDATMSILDISMMTGFAPDTKDLELLASGVDRYISKYEMNKAFSNKNTLIIYLEKISHTEEDCLTFKVHQYFNVGLIQPGSVKVYSYYNLEESCTRFYHPEKDDGMLSKLCHSEMCRCAEENCFMQQSQEKINLNVRLDKACEPGVDYVYKTELTNIELLDDFDEYTMTIQQVIKSGSDEVQAGQQRKFISHIKCRNALKLQKGKKYLMWGLSSDLWGEKPNTSYIIGKDTWVEHWPEAEECQDQKYQKQCEELGAFTESMVVYGCPN

SEQ ID NO: 3 - DNA, 5231 bp, Homo sapiens
  source 1..5231: mol_type = genomic DNA
actcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtg

SEQ ID NO: 4 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 5 - protein, 312 aa, Mus musculus
RLKHLIVTPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSCSSATTFR

SEQ ID NO: 6 - protein, 299 aa, Mus musculus
TPAGCGEQHMIGMTPTVIAVHYLDQTEQWGKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSRS

SEQ ID NO: 7 - protein, 293 aa, Mus musculus
TPAGCGEQHMIGMTPTVIAVHYLDQTEQWGKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSF

SEQ ID NO: 8 - protein, 302 aa, Homo sapiens
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 9 - DNA, 891 bp, Mus musculus
  source 1..891: mol_type = genomic DNA
acccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagaggccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagc

SEQ ID NO: 10 - protein, 297 aa, Mus musculus
TPAGCGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPS

SEQ ID NO: 11 - DNA, 894 bp, Homo sapiens
  source 1..894: mol_type = genomic DNA
accccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgc

SEQ ID NO: 12 - protein, 298 aa, Homo sapiens
TPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 13 - skipped

SEQ ID NO: 14 - DNA, 42 bp, synthetic construct
  source 1..42: mol_type = other DNA
  misc_feature 1..42: note = GSGGGGSGGGGSGS linker
ggctctggcgggggaggatcagggggtggcggctctggctca

SEQ ID NO: 15 - DNA, 15 bp, synthetic construct
  source 1..15: mol_type = other DNA
  misc_feature 1..15: note = GSEEF linker
ggctccgaggagttc

SEQ ID NO: 16 - DNA, 2757 bp, synthetic construct
  source 1..2757: mol_type = other DNA
  misc_feature 1..2757: note = C3d trimer
acccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagaggccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcggctctggcgggggaggatcagggggtggcggctctggctcaacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagaggccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagcggctctggcgggggaggatcagggggtggcggctctggctcaacccccgcaggctctggggaacagaacatgattggcatgacaccaacagtcattgcggtacactacctggaccagaccgaacagtgggagaagttcggcatagagaagaggcaagaggccctggagctcatcaagaaagggtacacccagcagctggccttcaaacagcccagctctgcctatgctgccttcaacaaccggccccccagcacctggctgacagcctacgtggtcaaggtcttctctctagctgccaacctcatcgccatcgactctcacgtcctgtgtggggctgttaaatggttgattctggagaaacagaagccggatggtgtctttcaggaggatgggcccgtgattcaccaagaaatgattggtggcttccggaacgccaaggaggcagatgtgtcactcacagccttcgtcctcatcgcactgcaggaagccagggacatctgtgaggggcaggtcaatagccttcctgggagcatcaacaaggcaggggagtatattgaagccagttacatgaacctgcagaggccatacacagtggccattgctgggtatgccctggccctgatgaacaaactggaggaaccttacctcggcaagtttctgaacacagccaaagatcggaaccgctgggaggagcctgaccagcagctctacaacgtagaggccacatcctacgccctcctggccctgctgctgctgaaagactttgactctgtgccccctgtagtgcgctggctcaatgagcaaagatactacggaggcggctatggctccacccaggctaccttcatggtattccaagccttggcccaatatcaaacagatgtccctgaccataaggacttgaacatggatgtgtccttccacctccccagc

SEQ ID NO: 17 - protein, 919 aa, synthetic construct
  REGION 1..919: note = C3d trimer
TPAGSGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSGSGGGGSGGGGSGSTPAGSGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPSGSGGGGSGGGGSGSTPAGSGEQNMIGMTPTVIAVHYLDQTEQWEKFGIEKRQEALELIKKGYTQQLAFKQPSSAYAAFNNRPPSTWLTAYVVKVFSLAANLIAIDSHVLCGAVKWLILEKQKPDGVFQEDGPVIHQEMIGGFRNAKEADVSLTAFVLIALQEARDICEGQVNSLPGSINKAGEYIEASYMNLQRPYTVAIAGYALALMNKLEEPYLGKFLNTAKDRNRWEEPDQQLYNVEATSYALLALLLLKDFDSVPPVVRWLNEQRYYGGGYGSTQATFMVFQALAQYQTDVPDHKDLNMDVSFHLPS

SEQ ID NO: 18 - DNA, 2802 bp, synthetic construct
  source 1..2802: mol_type = other DNA
  misc_feature 1..2802: note = C3d trimer
cacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcggctctggcgggggaggatcagggggtggcggctctggctcacacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcggctctggcgggggaggatcagggggtggcggctctggctcacacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgc

SEQ ID NO: 19 - protein, 934 aa, synthetic construct
  REGION 1..934: note = C3d trimer
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRGSGGGGSGGGGSGSHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRGSGGGGSGGGGSGSHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 20 - DNA, 3819 bp, Severe acute respiratory syndrome-related coronavirus
  source 1..3819: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagaaacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca

SEQ ID NO: 21 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARNLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - DNA, 648 bp, Severe acute respiratory syndrome-related coronavirus
  source 1..648: mol_type = genomic DNA
atgaagaccatcatcgccctgagctacatcttctgcctggtgttcgccactaatctttgtccgttcggtgaggtttttaacgcgacaaggttcgctagtgtatatgcttggaaccgaaagagaatctccaattgcgtagctgattactccgttctctataacagtgcgtccttttcaacctttaagtgttacggcgtttctccaacgaagctgaatgatctctgttttacgaacgtgtatgctgactctttcgttatacggggggacgaagtgagacagatagcaccaggtcagactgggaagatagcggattacaactataagttgcccgatgattttacggggtgcgtaatcgcatggaactcaaacaacctcgactccaaagtaggtggtaattataattacttgtatcgcctgtttcgaaagagcaatttgaagccttttgagcgggatatttcaaccgaaatttaccaagcaggcagtacgccatgtaacggagtagagggatttaattgctactttcctcttcaatcttatggctttcaaccaacaaacggagtggggtatcaaccttatagagtggtagtattgtcctttgagctcctccacgccccggctacagtttgtgggcccaaaaagggatccggt

SEQ ID NO: 23 - protein, 216 aa, Severe acute respiratory syndrome-related coronavirus
MKTIIALSYIFCLVFATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSG

SEQ ID NO: 24 - DNA, 648 bp, Severe acute respiratory syndrome-related coronavirus
  source 1..648: mol_type = genomic DNA
atgaagaccatcattgccctgagctacatcttctgcctggtgttcgccaccaacctgtgtcctttcggcgaggtgttcaacgccaccagattcgcctctgtgtacgcctggaacagaaagcggatcagcaactgcgtggccgactacagcgtgctgtacaacagcgccagcttcagcaccttcaagtgctacggcgtgtcccctaccaagctgaacgacctgtgcttcaccaacgtgtacgccgacagcttcgtgatcagaggcgacgaagtgcggcagatcgctcctggacagacaggcaagatcgccgattacaactacaagctgcccgacgacttcaccggctgtgtgatcgcttggaacagcaacaacctggacagcaaagtcggcggcaactacaactaccggtacaggctgttccgcaagagcaacctgaagcctttcgagagagacatcagcaccgagatctaccaggccggcagcaagccttgtaacggcgtggaaggcttcaactgctacttcccactgcagtcctacggcttccagcctacaaatggcgtgggctaccagccttacagagtggtggtgctgagcttcgagctgctgcacgctcctgctacagtgtgcggacctaagaaaggctctggc

SEQ ID NO: 25 - protein, 216 aa, Severe acute respiratory syndrome-related coronavirus
MKTIIALSYIFCLVFATNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKGSG

(19) United States
(12) Patent Application Publication
(10) Pub. No.: US 2023/0321230 A1
(43) Pub. Date: Oct. 12, 2023

(54) Compositions and Methods for Adjuvanted Vaccines

(71) Applicant: Massachusetts Institute of Technology, Cambridge, MA (US)

(72) Inventors: Bowen Li, Malden, MA (US); Allen Jiang, Cambridge, MA (US); Robert S. Langer, Newton, MA (US); Daniel G. Anderson, Framingham, MA (US)

(21) Appl. No.: 18/190,068

(22) Filed: Mar. 24, 2023

Related U.S. Application Data
(60) Provisional application No. 63/324,019, filed on Mar. 25, 2022.

(51) Int. Cl. A61K 39/215; C07K 14/47; A61K 39/39; C07K 14/005

(52) U.S. Cl. CPC A61K 39/39; A61K 39/215; C07K 14/005; C07K 14/472; A61K 2039/53

(57) ABSTRACT

Provided herein are, in various embodiments, methods and compositions comprising polynucleotides (e.g., mRNA) for eliciting an immune response. In certain embodiments, the disclosure provides for methods and compositions for enhancing efficacy of infectious disease treatment (e.g., mRNA vaccines). In still further embodiments, the disclosure provides methods and compositions for enhancing one or more vaccines, such as SARS-CoV-2 mRNA vaccines.

### RELATED APPLICATION

This application claims the benefit of U.S. Provisional Application No. 63/324,019, filed on Mar. 25, 2022. The entire teachings of the above application is incorporated herein by reference.

### INCORPORATION BY REFERENCE OF MATERIAL IN XML

This application incorporates by reference the Sequence Listing contained in the following eXtensible Markup Language (XML) file being submitted concurrently herewith:

a) File name: 00502369001.xml; created Mar. 22, 2023, 55,828 Bytes in size.

### GOVERNMENT SUPPORT

This invention was made with government support under UG3 HL147367 and 5R61AI161805 from National Institutes of Health. The government has certain rights in the invention.

### BACKGROUND

The Emergency Use Authorizations of the Pfizer-BioNTech (BNT162b2) and Moderna mRNA-1273 vaccines represent a crucial step towards altering the course of the severe acute respiratory syndrome coronavirus 2 (SARS-CoV-2) pandemic. These authorizations have also demonstrated the promising potential of mRNA as a new vaccine class for combating infectious diseases. The ability to design and synthesize mRNA vaccines encoding a wide variety of antigens rapidly, without the costly bioreactors required for conventional vaccines, is particularly advantageous. Unlike DNA vaccines, mRNA vaccines only need to enter the cell’s cytoplasm to enable antigen expression and there is no risk of genomic integration.

The widespread clinical translation of this technology requires overcoming certain limitations. Accordingly, there is a need in the art for enhancing mRNA vaccines.

### SUMMARY

In one aspect, the present disclosure provides for a polynucleotide construct comprising a first polynucleotide sequence encoding an agent; and a second polynucleotide encoding a C3 complement protein degradation product (C3d) or a fragment thereof; wherein the first polynucleotide is operably connected to the second polynucleotide. Other aspects include nanoparticles and compositions comprising one or more constructs, along with methods of making them.

In another aspect, the present disclosure provides for a method of inducing a response to an antigen in a cell, the method comprising contacting the cell with a composition comprising: a first polynucleotide sequence encoding an agent, and a second polynucleotide sequence encoding a C3 complement protein degradation product (C3d) or a fragment thereof; wherein the first polynucleotide sequence is operably connected to the second polynucleotide sequence; and wherein the response is induced after contact with the composition.

In another aspect, the present disclosure provides for a method of eliciting an enhanced immune response in a subject, the method comprising administering to the subject a composition comprising a first polynucleotide sequence encoding an agent, and a second polynucleotide sequence encoding a C3 complement protein degradation product (c3d) or a fragment thereof; wherein the first polynucleotide sequence is operably connected to the second polynucleotide sequence; and wherein the subject exhibits an enhanced immune response after administration of the composition. In some aspects, the disclosure provides for embodiments wherein the agent is an immunogen, a peptide, an antigen, an antibody, or combination thereof.

In another aspect, the present disclosure provides for a method of treatment. In certain embodiments, the treatment is for an infectious disease. In some embodiments, the infectious disease is a coronavirus (e.g., Severe acute respiratory syndrome-related coronavirus), influenza virus, respiratory syncytial virus (RSFV), human immunodeficiency virus, zika virus, Epstein-Barr virus, herpes simplex virus, rabies, cytomegalovirus, mycobacterium tuberculosis, or a combination thereof.

In still other aspects, the disclosure provides for a method of treating cancer. In certain embodiments, the cancer is melanoma, colorectal cancer, high-risk melanoma, human papilloma virus, head and neck squamous carcinoma, non-small cell lung cancer, New York esophageal squamous cell carcinoma, or a combination thereof.

In further aspects, the disclosure provides for a composition comprising a polynucleotide construct. In some embodiments, the polynucleotide construct comprises a first polynucleotide sequence encoding an agent; and a second polynucleotide encoding a C3 complement protein degradation product (C3d) or a fragment thereof; wherein the first mRNA is operably connected to the second mRNA.

Further, the present disclosure provides for methods of making and using the methods and compositions disclosed herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A shows a schematic of the design of SARS-Cov-2 spike protein (SP) fused with three copies of C3 complement protein degradation product (C3d), a component of complement system for vaccination. FIGS. 1B and 1C show titers of anti-SP IgG detected in sera of C57BL/6J mice that were immunized intramuscularly with: LNP-formulated firefly luciferase mRNA (FFL, negative control); LNP-formulated mRNA encoding SARS-CoV-2 spike protein (SP); LNP-formulated mRNA encoding C3d-fused SARS-CoV-2 spike protein (SP-C3d); and mRNA encoding SARS-CoV-2 spike protein encapsulated within a lipopolysaccharide (LPS/SP) at concentrations ranging from 0.01 µg-1 µg on day 0 (prime) and day 21 (boost); n = 5. FIG. 1B shows titers of anti-SP IgG detected in sera of mice collected on day 14 post prime. FIG. 1C shows titers of anti-SP IgG detected in sera collected on day 35 post prime. FIG. 1D shows ELISpot assay of IFN-γ-spot-forming cells among splenocytes after ex vivo re-stimulation with SARS-CoV-2 peptides in different nanoparticle-treated groups, expressed as spot forming units (SFU) per 2.5×10⁵ cells (n = 5). FIG. 1E shows SARS-CoV-2 SP-specific CD4+ Tem cells (CD44+CD62L-) in splenocytes detected by flow cytometry (n = 5). FIG. 1F shows SARS-CoV-2 SP-specific MFI CD21 on B cells (B220+) in splenocytes detected by flow cytometry (n = 5). FIG. 1G shows titers of anti-RBD_{delta} IgG detected in sera on day 35 post prime of C57BL/6J mice that were immunized with ionizable lipid nanoparticles (LNPs) formulated with RBD_{delta} mRNA, RBD_{delta}-C3d fusion mRNA, and RBD_{delta}/C3d mRNA mixture at the dose of 1 µg on day 0 (prime) and day 21 (boost); n = 4-5. FIG. 1H shows levels of different anti-RBD_{delta} Abs in sera characterized by a multiplexed method. The heatmap shows the z-score for each feature against RBD_{delta} in PBS- and different LNP-treated groups. For FIGS. 1B-1G, statistical significance was analyzed by a two-tailed Student’s t-test. Data are presented as mean±SD.

FIG. 2 shows titers of anti-RBD_{delta} IgG in sera on day 35 post prime of C57BL/6J mice immunized with MC3 LNPs formulated with RBD_{delta} mRNA or RBD_{delta}-C3d fusion mRNA at the dose of 1 µg by either intranasal (IN) or intramuscular (IM) administration. (n = 4-5).

FIG. 3A shows an analysis of mRNA by gel electrophoresis. 1.5 µg of mRNA was loaded into each lane. FIG. 3B shows SP, SP-C3d, RBD, and RBD-C3d expression in transfected HEK293F cells determined by ELISA following transfection with 1 µg of mRNA. FIG. 3C shows fluorescent western blot of denatured lysate samples from HEK293T cells transfected with mSP or mSP-C3d lysed at either 6 h or 24 h following transfection. Detection was performed by first incubating blots with rabbit anti-actin, rabbit anti-S2, and goat anti-C3d followed by incubation with secondary antibodies against rabbit and goat IgGs. FIG. 3D shows chemiluminescent western blot of denatured lysate samples from HEK293T cells transfected with mRBD, mRBD-C3d, or mC3d. All transfected groups run in duplicate with each lane representing an individual well of HEK293T cells transfected with mRNA. Detection was performed with antibodies against actin, RBD, and C3d.

FIG. 4A shows encapsulation efficiency (EE%) of LNPs formulated with different mRNA encoding SP, SP-C3d, RBD, and RBD-C3d respectively. FIG. 4B shows particle size of LNPs formulated with different mRNA encoding SP, SP-C3d, RBD, or RBD-C3d, respectively.

FIG. 5A shows firefly luciferase mRNA (FFL) expression by optical imaging at 6 h after LNPs formulated with FFL encoding mRNA were injected intramuscularly into mice (0.25 mg/kg mRNA). FIG. 5B shows FFL expression by optical imaging at 24 h after LNPs formulated with FFL encoding mRNA were injected intramuscularly into mice (0.25 mg/kg mRNA).

FIGS. 6A and 6B show titers of anti-RBD IgG detected in sera of C57BL/6J mice that were immunized intramuscularly with: LNP-formulated firefly luciferase mRNA (FFL); LNP-formulated mRNA encoding SARS-CoV-2 receptor binding domain (RBD); and LNP-formulated mRNA encoding C3d-fused SARS-CoV-2 receptor binding domain (RBD-C3d) at concentrations ranging from 0.01 µg-1 µg on day 0 (prime) and day 21 (boost); n = 5 biologically independent mice per group. FIG. 6A shows titers of anti-RBD IgG detected on day 14 post prime. FIG. 6B shows titers of anti-RBD IgG detected on day 35 post prime.

FIGS. 7A-7C show a gating strategy of SARS-CoV-2 RBD-specific CD4+ T effector memory (Tem) cells (CD44+CD62L) using flow cytometry analysis. FIG. 7A shows the whole population of lymphocytes; FIG. 7B shows CD4+/- and CD8+/- cells among the lymphocytes in FIG. 7A; and FIG. 7C shows CD44+/- and CD62L+/- cells among the CD4+CD8- cell population in FIG. 7B.

FIG. 8A shows concentration changes of systemic cytokines and chemokines in mouse sera at 6 h post immunization of SARS-CoV-2 vaccines (1 µg mRNA per mouse) compared to that in untreated mouse sera (n=5, each column represents an individual mouse). FIG. 8B shows MFIs of IgG subclasses obtained from Luminex assay measuring serological antibody binding against the RBD antigen from the Delta variant of SARS-CoV-2. Data related to FIG. 1H. FIG. 8C shows ratio of IgG2c to IgG1 levels as a surrogate of Th1-Th2 bias. Ratios were calculated as log₁₀(MFI_{IgG2c})/log₁₀(MFI_{IgG1}). n=5, statistical significance was analyzed by using two-way ANOVA with post-hoc Tukey test.

FIG. 9 shows polar plots show the mean percentile rank for each antibody feature against RBD from the Delta variant of SARS-CoV-2 in serum collected from mice two weeks post-boost vaccination. Data are related to vaccination study in FIG. 2.

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines, and animals. The present disclosure is not limited by the illustrated ordering of acts or events, as some acts may occur in different orders and/or concurrently with other acts or events. Furthermore, not all illustrated acts, steps, or events are required to implement a methodology in accordance with the present disclosure. Many of the techniques and procedures described, or referenced herein, are well understood and commonly employed using conventional methodology by those skilled in the art.

Unless otherwise defined, all terms of art, notations, and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

As used herein, the indefinite articles “a,” “an,” and “the” should be understood to include plural reference unless the context clearly indicates otherwise.

Throughout this specification and the claims which follow, unless the context requires otherwise, the word “comprise,” and variations such as “comprises” and “comprising,” will be understood to imply the inclusion of, e.g., a stated integer or step or group of integers or steps, but not the exclusion of any other integer or step or group of integers or steps. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary the scope of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

The successful development of mRNA-based vaccines against SARS-CoV-2 has provoked broad interest in RNA-based technologies. However, widespread clinical translation of these mRNA-based therapies requires overcoming certain limitations.

One such limitation is that the mRNA must be delivered into the cytoplasm, overcoming barriers such as endogenous RNase enzymes and the cell membrane, in order to achieve protein expression. To address the delivery challenge and improve in vivo protein expression, nanoparticle technologies using polymer and lipid-like systems such as lipid nanoparticles (LNPs) as carriers may be employed. Another challenge is then to elicit a sufficiently potent immune response at a safe and tolerable dose. This can be achieved through the addition of adjuvants in the formulation, which potentiate the immune response.

Additionally, improving antigen specific immune responses through adjuvant addition can lower the dose required to cross a protective immunity threshold, reducing the cost associated with mRNA therapy production, increasing the number of doses available for clinical distribution, and potentially limiting side effects associated with administration.

For mRNA vaccine formulations, localization of an adjuvant with the mRNA transcript can improve antigen-specific immune response while avoiding undesirable systemic activation of the immune system. One strategy for adjuvant and mRNA co-localization has been utilization of encapsulating nanoparticles as the adjuvant.

An alternative approach is through design of the mRNA transcript itself. Accordingly, the present disclosure provides for immuno-modulatory proteins which may be easily incorporated into an mRNA transcript encoding an agent of interest.

As provided herein, the present disclosure shows that administration of mRNA protective and/or therapeutic compositions, including vaccines encoding for an antigen-C3d fusion induce a ten-fold higher level of antibodies in mice following a prime-boost vaccination strategy when compared to the same mRNA vaccines without C3d.

Analysis of cellular responses and cytokine profiles reveal that inclusion of C3d may enhance the Th1 immune response which is favorable for avoiding a Th2 immune response linked with vaccine-associated enhanced respiratory disease. As such, the present disclosure provides evidence for the promising potential of C3d incorporation into a mRNA vaccine transcript to produce a new class of self-adjuvanted mRNA vaccines. This self-adjuvanted approach is more effective in inducing adaptive immune responses and allows precise spatial control of antigen and adjuvant as well as tunable immune stimulation.

Given that the U.S. government has considered giving some people half the dose of COVID-19 vaccine in order to accelerate vaccinations, this advantageous antigen-C3d fusion is highly meaningful to expediate the clinical expansion of SARS-CoV-2 vaccines. A low dose of vaccines enabled by antigent-C3d fusion could not only reduce the cost of vaccination but may also prevent the occurrence of unexpected sides effects, improving the safety of mRNA vaccines.

As a highly versatile platform that enhances the immunogenicity of mRNA vaccines via molecular design, the present disclosure provides for a self-adjuvanted mRNA platform that can be readily delivered, for example, using current LNP formulation, saving the time and cost spent on formulation screening or optimization. In some embodiments, the present disclosure provides means to improve the efficacy and/or the potency of mRNA vaccines. It is expected to find wide application in the development of mRNA treatments for infectious diseases and oncology.

Given the current global health crisis due to SARS-CoV-2, the present disclosure employs a C3d-based mRNA adjuvant approach using the spike protein (SP) and receptor binding domain (RBD) of SARS-CoV-2 as model antigens. Accordingly, the present disclosure further demonstrates that the disclosed C3d adjuvating strategy is capable of increasing antibody titers to the SARS-CoV-2 Delta variant RBD. Additionally, this potentiation of immune response was observed for both intramuscular and intranasal routes of administration.

Safe and effective mRNA vaccines require both intracellular mRNA delivery and controlled adjuvancy to produce an optimal vaccine response. The present disclosure develops a multiply-adjuvanted mRNA vaccine system whereby the mRNA encoded antigen is engineered to potentiate the immune response. Using cues from natural immunity, a modular platform for adjuvanting the antigen encoded by mRNA was developed by creating a fusion protein consisting of an antigen of interest and a natural adjuvant derived from C3 complement protein (C3d). Compared to conventional mRNA-encoded antigen, fusion with C3d increases the induction of anti-SARS-CoV-2 antibody titers by ten-fold for both wild-type and Delta virus antigens. These multiply-adjuvanted mRNA vaccines have the potential to improve mRNA vaccines’ efficacy, safety, and ease of administration.

The FDA approvals of Pfizer/BioNTech’s BNT162b2 and Moderna’s mRNA-1273 represent a crucial step toward altering the course of the SARS-CoV-2 pandemic. They have also demonstrated the promising potential of mRNA as a new vaccine class for combating infectious diseases. The ability to rapidly design and synthesize mRNA vaccines encoding a wide variety of antigens without the costly bioreactors required for conventional vaccines is particularly advantageous. Unlike DNA vaccines, mRNA vaccines only need to enter the cell’s cytoplasm to enable antigen expression, and there is no risk of genomic integration.

Although two mRNA vaccines have been authorized for use against SARS-CoV-2 and many more mRNA vaccines for other infectious diseases are in clinical trials, the widespread clinical translation of this technology requires overcoming certain limitations. First, the mRNA must be delivered into the cytoplasm, overcoming barriers such as endogenous RNAse enzymes and the cell membrane to achieve protein expression. To address this delivery challenge and improve in vivo protein expression, nanoparticle technologies using polymer and lipid-like systems such as lipid nanoparticles (LNPs) as carriers have been widely utilized. The second challenge is eliciting an appropriate immune response to the expressed antigen at a tolerable dose. While existing mRNA vaccines do not have additional adjuvants added, this is a common component of conventional protein vaccines. Improving antigen-specific immune responses through adjuvant addition can lower the dose required to cross a protective immunity threshold. This can reduce the cost associated with mRNA vaccine production, increasing the number of doses available for clinical distribution and potentially limiting side effects related to administering the vaccine formulation.

For mRNA vaccine formulations, localization of an adjuvant with the mRNA transcript can improve antigen-specific immune response while avoiding undesirable systemic activation of the immune system. Although unmodified, and to some extent modified, mRNA itself may possess adjuvant characteristics due to its ability to stimulate innate immune responses through toll-like receptor or RIG-I sensing or through alternative sensing pathways which lead to the production of IL-1R associated cytokines; stimulation of these pathways may also lead to reactogenic responses.

The present strategy for integrating an adjuvant into a nanoparticle is through the design of the mRNA transcript itself. As described herein, naturally occurring immuno-modulatory proteins may be easily incorporated into the same mRNA transcript that encodes the antigen of interest. C3d is the terminal degradation product of mammalian complement component C3, a protein of the innate immune system. Activation of complement can lead to covalent attachment of C3d to the activating antigen. Interaction between C3d and CD21, the C3d receptor on B cells and follicular dendritic cells (FDCs), leads to strong B cell stimulation, improved antigen presentation on FDCs, and subsequently robust robustness lymphocyte activation. Thus, delivery of an antigen-C3d fused mRNA vaccine using could provoke a more robust immune response when compared to the same mRNA vaccine without C3d. The addition of C3d to an mRNA vaccine should not affect the incorporation of the mRNA into existing nanoparticle and/or nanocarrier formulations because the adjuvant is directly integrated into the mRNA transcript.

While most mRNA vaccines, and traditional vaccines more broadly, are currently administered intramuscularly (IM), there is growing interest in intranasal (IN) administration of mRNA vaccines, given their advantages of needle-free delivery and the ability to induce local mucosal immunity in the nasal and bronchial airways which may be particularly effective at protecting against respiratory diseases like SARS-CoV-2. Unlike IM vaccinations which mainly generate a serum IgG response, intranasal vaccinations or infections are reported to elicit an IgA response in the mucosal linings of the nasal and upper airways, with these secreted IgAs shown to strongly neutralize respiratory viruses. Additionally, intranasal vaccinations can elicit tissue-resident memory B and T cell localization in the nose and lung, acting more rapidly as cellular first responders to respiratory infection than systemic memory cells. While preclinical studies of intranasal mRNA vaccines have been reported, the role that adjuvants play in potentiating the immune response following intranasal mRNA vaccinations has not been explored.

Given the current global health crisis due to SARS-CoV-2, described herein is a C3d-based mRNA adjuvant approach using the spike protein (SP) and receptor-binding domain (RBD) of SARS-CoV-2 as model antigens. The present disclosure shows that administration of mRNA vaccines encoding for the antigen-C3d fusion induces a ten-fold higher level of antibodies in mice following a prime-boost vaccination strategy than the same mRNA vaccines without C3d. It is further demonstrated that the C3d adjuvanted approach can also increase antibody titers to the SARS-CoV-2 Delta variant RBD. The combination of the adjuvanted ionizable lipid and mRNA transcript vaccination strategies resulted in a synergistic potentiation of immune responses to the Delta variant of SARS-CoV-2 and was observed for intramuscular and intranasal routes of administration.

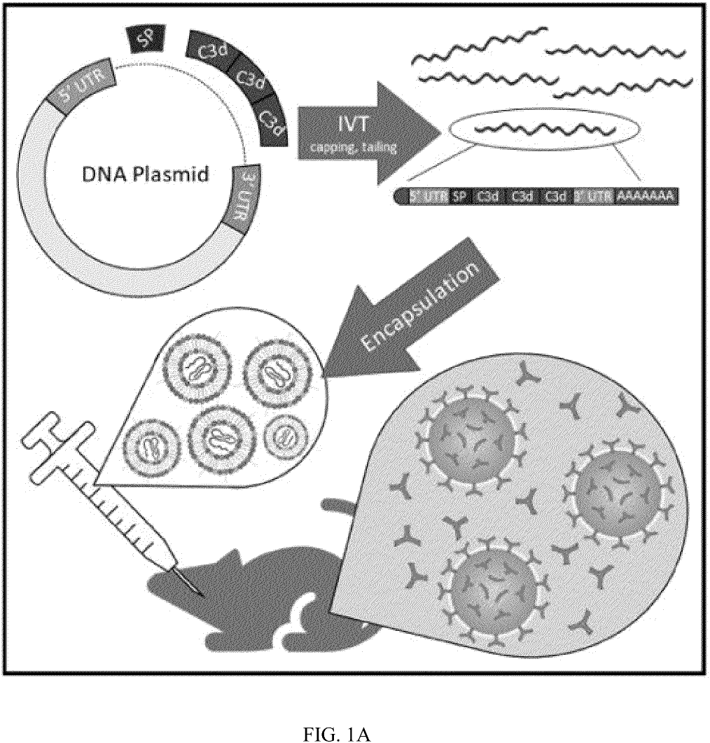

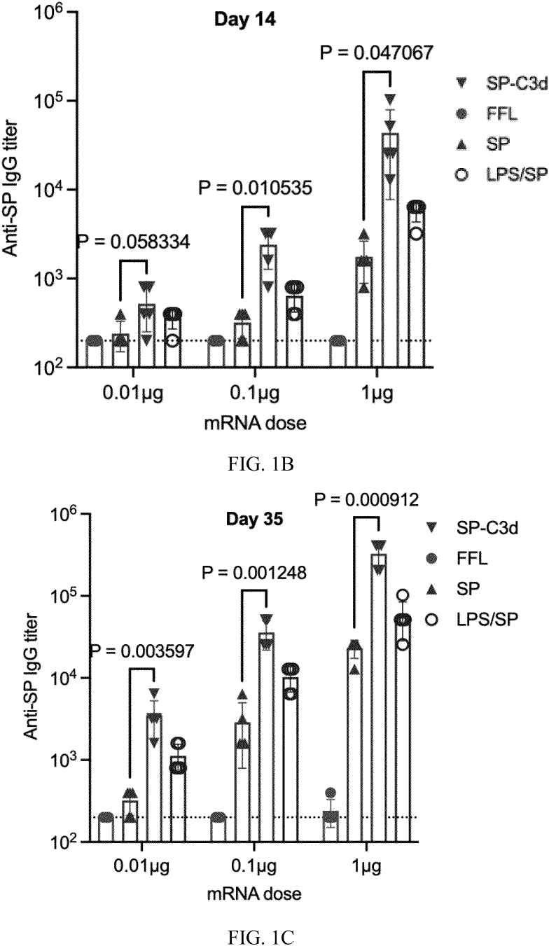

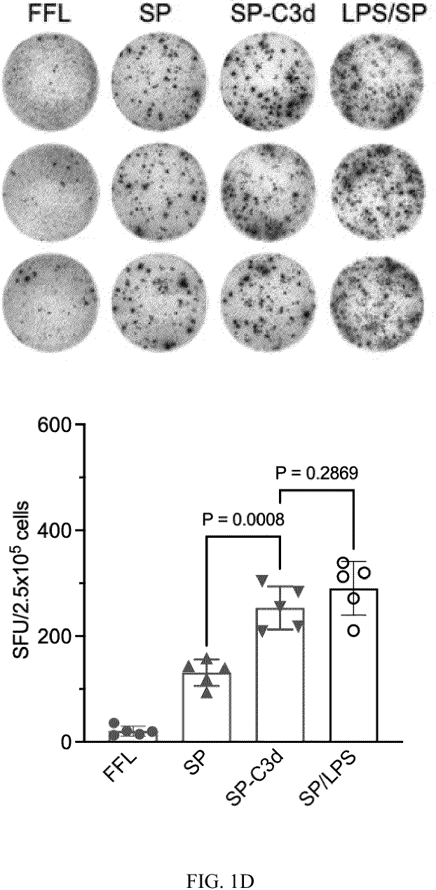

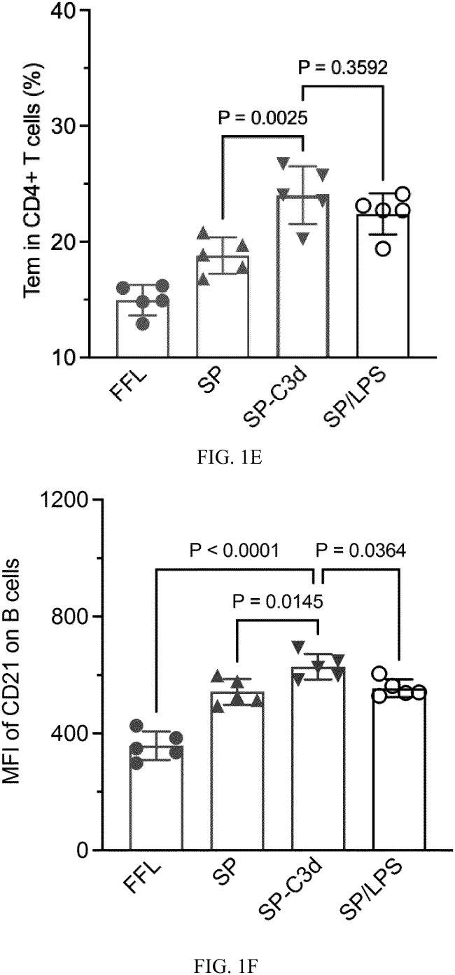

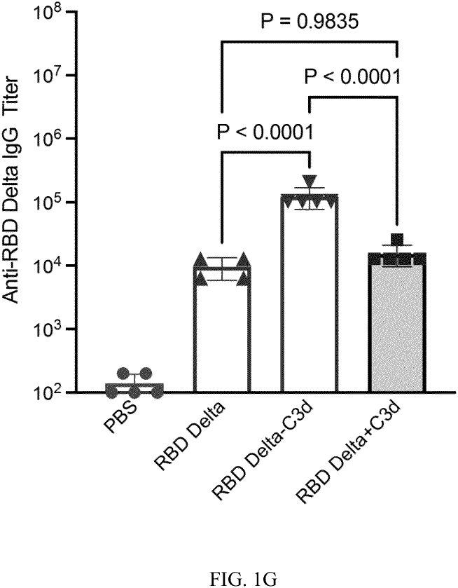

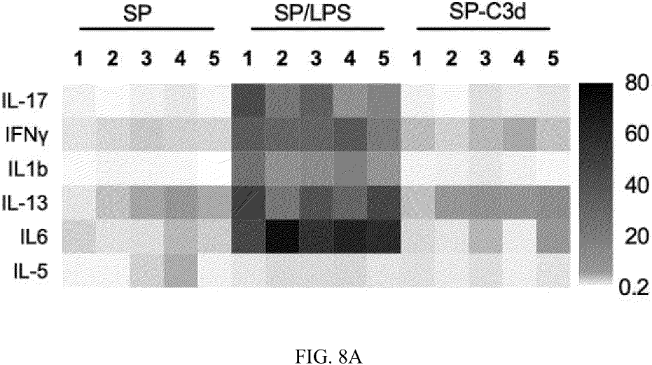

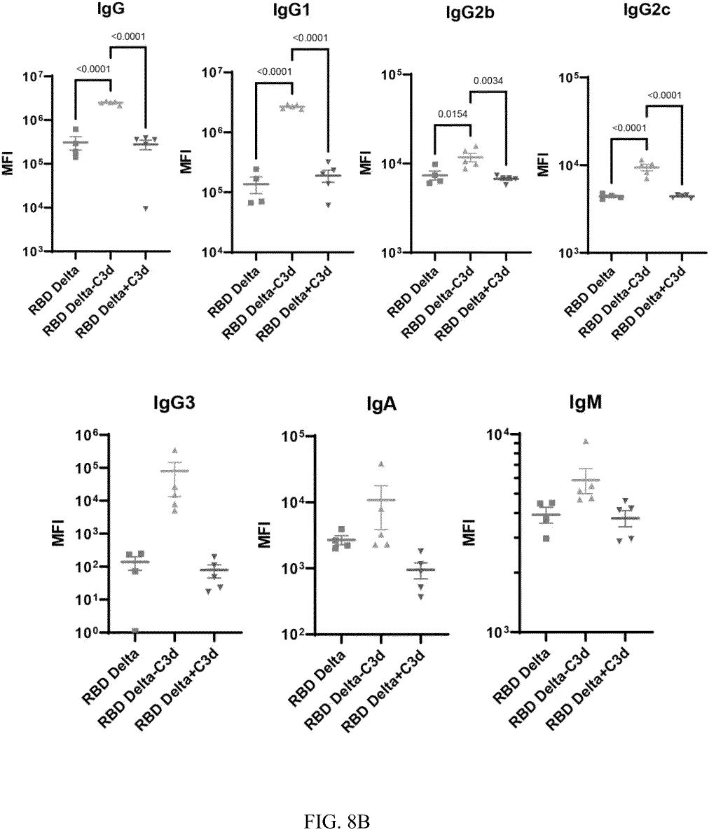

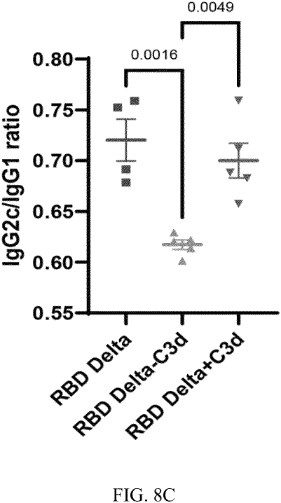

Despite adjuvant effects of C3d shown in protein and DNA vaccine studies, the incorporation of C3d in an mRNA vaccine has not been previously demonstrated. Using SARS-CoV-2 SP and RBD as model antigens, it is demonstrated herein that two immunizations of mRNA encoding SP-C3d or RBD-C3d can induce 10-fold higher titers of antibodies than that induced by mRNA-encoding only SP or RBD. It is further disclosed herein that that the C3d fusion could also be applied for vaccination against the Delta variant of SARS-CoV-2. It is expected that the C3d fusion strategy can also be used to develop mRNA vaccines against other variants of concern, including the Omicron variant. Analysis of cellular responses to the C3d vaccine strategy indicates that the C3d fusion can enhance the release of IFN-ɤ (FIG. 1D) associated with a TH1 immune response and improved disease outcome. Compared with classic strategies that involve co-administration of the mRNA transcript with LPS, this adjuvant approach is more effective in inducing adaptive immune responses. Also, it allows precise spatial control of antigen and adjuvant stimulation to avoid undesirable elevation of systemic, inflammatory cytokine levels associated with reactogenicity of mRNA vaccines (FIG. 8A).

### Methods of the Disclosure

In one aspect, the present disclosure provides a method of inducing a response to an antigen, e.g., in a cell, the method comprising contacting the cell with a composition, construct or nanoparticle comprising a first polynucleotide sequence encoding an agent, and a second polynucleotide sequence encoding a C3 complement protein degradation product (C3d) or a fragment thereof; wherein the first polynucleotide sequence is operably connected to the second polynucleotide sequence; and wherein the response is induced after contact with the composition, construct or nanoparticle.

In one aspect, the present disclosure provides a method of eliciting an enhanced immune response in a subject, the method comprising the step of administering to the subject a composition comprising: a first polynucleotide sequence encoding an agent, and a second polynucleotide sequence encoding a C3 complement protein degradation product (C3d) or a fragment thereof; wherein the first polynucleotide sequence is operably connected to the second polynucleotide sequence; and wherein the subject exhibits an enhanced immune response after administration of the composition.

As used herein, “subject” or “patient” includes humans, domestic animals, such as laboratory animals (e.g., dogs, monkeys, pigs, rats, mice, etc.), household pets (e.g., cats, dogs, rabbits, etc.) and livestock (e.g., chickens, pigs, cattle (e.g., a cow, bull, steer, or heifer), sheep, goats, horses, etc.), and non-domestic animals. In some embodiments, a subject is a mammal (e.g., a non-human mammal). In some embodiments, a subject is a human. In still further embodiments, a subject of the disclosure may be a cell, cell culture, tissue, organ, or organ system.

An “immune response” to an agent or composition is the development in a subject of a humoral and/or a cellular immune response to an agent present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

A composition or vaccine that elicits a cellular immune response may serve to sensitize a subject by the presentation of antigen in association with MHC molecules at the cell surface. The cell-mediated immune response is directed at, or near, cells presenting antigen at their surface. In addition, antigen-specific T-lymphocytes can be generated to allow for the future protection of an immunized host.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immol. (1994) 24:2369-2376. Recent methods of measuring cell-mediated immune response include measurement of intracellular cytokines or cytokine secretion, or by measurement of the relative IgG concentration. In some embodiments, immune response is measured using longevity of immunity, percent reduction in risk of disease cases in a population of subjects administered the composition, reduction of relative risk (RR) of disease among a population of subjects administered the composition, transmissibility, or a combination thereof.

As used herein, the term “enhanced” when used with respect to an immune response, such as a CD4+ T cell response, an antibody response, or a CD8+ T cell response, refers to an increase in the immune response in a subject administered a composition of the present disclosure, relative to the corresponding immune response observed from a subject prior to administration and/or the corresponding immune response observed from a subject administered a control composition.

As used herein, “administering” or “administration” refers to taking steps to deliver a composition to a subject. Administering can be performed, for example, once, a plurality of times, and/or over one or more extended periods. Administration includes both direct administration, including self-administration, and indirect administration, including the act of prescribing or directing a subject to consume a composition. For example, as used herein, one (e.g., a physician) who instructs a subject (e.g., a patient) to self-administer a composition (e.g., a drug), or to have the composition administered by another and/or who provides a subject with a prescription for a composition is administering the composition to the subject.

As used herein, in certain aspects, the unexpected or enhanced immune response in a subject disclosed herein can protect the subject against various diseases and/or infections (e.g., against bacterial and/or viral diseases). In some embodiments, compositions of the disclosure are immunogenic, and are vaccine compositions. Vaccines according to the disclosure may either be prophylactic (i.e., to prevent infection) or therapeutic (i.e., to treat infection),

In some embodiments, the present disclosure provides for a composition comprising a first polynucleotide sequence encoding an agent, e.g., an agent associated with an infectious disease. In other embodiments, the present disclosure provides for compositions comprising an agent associated with cancer. In certain embodiments, the agent is an immunogen, a peptide, an antigen, an antibody, or combination thereof.

### Degradation Product of C3 Complement Proteins

In one aspect, the present disclosure provides for improved performance of SARS-Cov-2 mRNA vaccines, comprising a self-adjuvanted mRNA vaccine system. Given the current global health crisis due to SARS-CoV-2, in one aspect, the present disclosure provides for a C3d-based adjuvant approach using the spike protein (SP) and/or the ribosome binding domain (RBD) of SARS-CoV-2 as antigens.

As disclosed herein, delivery of a self-adjuvanted, antigen-C3d fused mRNA vaccine using an LNP formulation provokes a stronger immune response when compared to the same mRNA vaccine without C3d. Compared to the mRNA encoding viral proteins alone, the inclusion of a C3d trimer increases the magnitude of antigen-specific antibody titers by at least ten-fold in mouse sera. Analysis of cellular responses and cytokine profiles reveal that the C3d fusion further skews the immune response to Th-1 phenotype, favorable to avoid the Th-2 biased response linked with vaccine-associated enhanced respiratory disease. Moreover, this self-adjuvanted approach causes much lower systemic cytokine expression than classic adjuvant strategies that involve co-administration of adjuvant with the mRNA transcript. Hence, the C3d-fusion mRNA system may reduce the minimum dosage for mRNA vaccines to induce sufficient immunity and is anticipated to find wide applications in infectious diseases and oncology therapeutics.

As used herein, a “C3 complement protein degradation product” or “C3d” is a terminal degradation product of mammalian complement component C3, a protein of the innate immune system. Activation of complement can lead to covalent attachment of C3d to the activating antigen. Interaction between C3d and CD21, the C3d receptor present on B cells, leads to strong B cell stimulation and subsequently robust lymphocyte activation.

In certain embodiments, C3 protein is Mus musculus C3 (NCBI Ref. Seq. No. NM_009778.3) and is encoded by the sequence of SEQ ID NO: 1. In some embodiments, the encoded Mus musculus C3 (NCBI Ref. Seq. No. NP_033908.2) amino acid sequence is given by the sequence of SEQ ID NO: 2.

In other embodiments, the C3 protein is human (NCBI Ref. Seq No. NM_000064.4) and is encoded by the sequence of SEQ ID NO: 3. In some embodiments, the encoded human C3 (NCBI Ref. Seq. No. NP_000055.2) amino acid sequence is given by the sequence of SEQ ID NO: 4.

In certain embodiments, the C3d is murine C3d. In some embodiments the murine C3d is UniProt KB E1APH6 comprising the sequence of SEQ ID NO:5. In some embodiment the murine C3d is UniProt KB Q207D2 comprising the sequence of SEQ ID NO:6. In still further embodiments the murine C3d is UniProt KB B5APU1 comprising the sequence of SEQ ID NO: 7. In still further embodiments, the C3d is human and comprises the sequence of SEQ ID NO: 8. In some embodiments, the C3 complement protein degradation product (C3d) or fragment thereof comprises the sequence of SEQ ID NO: 10 or SEQ ID NO: 12.

In still further embodiments, the C3d or fragment thereof is at least about 70% identical (i.e., comprises at least about 70% sequence identity) to SEQa ID NO: 5, SEQ ID NO: 6, SEQ ID NO: 7, SEQ ID NO: 8, SEQ ID NO: 10, or SEQ ID NO: 12, for example, has at least about: 75%, 80%, 85%, 90%, 95%, 96%, 97%, 98%, 99% or 100% sequence identity to SEQ ID NO: 5, SEQ ID NO: 6, SEQ ID NO: 7, SEQ ID NO: 8, SEQ ID NO: 10, or SEQ ID NO: 12. In certain embodiments, the C3d or fragment thereof comprises an amino acid sequence that is about: 70%, 75%, 80%, 85%, 90%, 95%, 96%, 97%, 98%, 99% or 100% identical to SEQ ID NO: 5, SEQ ID NO: 6, SEQ ID NO: 7, SEQ ID NO: 8, SEQ ID NO: 10, or SEQ ID NO: 12. In some embodiments, the C3d or fragment thereof comprises an amino acid sequence having about 70%, 75%, 80%, 85%, 90%, 95%, 96%, 97%, 98%, 99% or 100% sequence identity to SEQ ID NO: 5, SEQ ID NO: 6, SEQ ID NO: 7, SEQ ID NO: 8, SEQ ID NO: 10, or SEQ ID NO: 12. In particular embodiments, the C3d or fragment thereof comprises a sequence having about 70-100% sequence identity to SEQ ID NO: 5, SEQ ID NO: 6, SEQ ID NO: 7, SEQ ID NO: 8, SEQ ID NO: 10, or SEQ ID NO: 12, for example, about: 75-100%, 75-99%, 80-100%, 80-98%, 85-100%, 85-97%, 90-100%, 90-96%, 95-100%, 96-100%, 97-100%, 98-100% or 99-100%.

In still further embodiments, the C3d or fragment thereof is encoded by a polynucleotide sequence. In some embodiments, the polynucleotide is at least about 70% identical (i.e., comprises at least about 70% sequence identity) to SEQ ID NO: 9 or SEQ ID NO: 11, for example, has at least about: 75%, 80%, 85%, 90%, 95%, 96%, 97%, 98%, 99% or 100% sequence identity to SEQ ID NO: 9 or SEQ ID NO: 11. In certain embodiments, the polynucleotide comprises a nucleotide sequence that is about: 70%, 75%, 80%, 85%, 90%, 95%, 96%, 97%, 98%, 99% or 100% identical to SEQ ID NO: 9 or SEQ ID NO: 11. In some embodiments, the polynucleotide comprises a nucleotide sequence having about 70%, 75%, 80%, 85%, 90%, 95%, 96%, 97%, 98%, 99% or 100% sequence identity to SEQ ID NO: 9 or SEQ ID NO: 11. In particular embodiments, the polynucleotide comprises a nucleotide sequence having about 70-100% sequence identity to SEQ ID NO: 9 or SEQ ID NO: 11, for example, about: 75-100%, 75-99%, 80-100%, 80-98%, 85-100%, 85-97%, 90-100%, 90-96%, 95-100%, 96-100%, 97-100%, 98-100% or 99-100%.

In some embodiments, the C3d or fragment thereof is an RNA (e.g., mRNA) polynucleotide sequence. In some embodiments, the RNA polynucleotide sequence is selected from the group consisting of SEQ ID NO: 9, SEQ ID NO: 11, and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 9 and SEQ ID NO: 11. In some embodiments, the RNA polynucleotide sequence is encoded by at least one fragment of a nucleic acid sequence (e.g., a fragment having an antigenic sequence or at least one epitope) sequence is selected from the group consisting of SEQ ID NO: 9, SEQ ID NO: 11, and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 9 and SEQ ID NO: 11.

As used herein, the term “sequence identity,” refers to the extent to which two sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat′l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology). In some embodiments, codon-optimized sequences for efficient expression in different cells, tissues, and/or organisms reflect the pattern of codon usage in such cells, tissues, and/or organisms containing conservative (or non-conservative) amino acid substitutions that do not adversely affect normal activity.

As used herein, “operably linked” or “operable connected” refers to an arrangement of elements wherein the components so described are configured so as to perform their usual function. Thus, in some embodiments, a control element operably linked to a coding sequence is capable of effecting the expression of the coding sequence. In other embodiments, one or more elements are operably linked such that the operably linked elements are in-frame wherein an open reading frame encodes a single polypeptide.

In some embodiments, a polynucleotide of the disclosure encodes a polymer. In some embodiments, a polynucleotide according the disclosure is a multimer. In still further embodiments, the multimer is a dimer, trimer, or tetramer. Polypeptides may comprise a single chain or multichain polypeptides.

As used herein, the arrangement of elements to form an operable connection may further comprise a linker. In some embodiments, a “linker” refers to a short amino acid sequence between two and 25 amino acids, although longer linkers are also contemplated. In some embodiments, a “linker” refers to a short nucleic acid sequence between six and 75 nucleotides, although longer linkers are also contemplated. In certain embodiments, the linker is encoded by a sequence selected from the group consisting of SEQ ID NO: 13 (ggctca), SEQ ID NO: 14, and SEQ ID NO: 15. In still further embodiments, a “linker” may be a chemical linking group that is covalently bonded to one or more elements. In certain embodiments of the present disclosure, linkers are flexible, permitting the attachment of two elements, without the disrupting the structure, aggregation or activity of the individual elements.

Accordingly, in certain embodiments of the disclosure, the polynucleotide is a C3d trimer. In some embodiments, the C3d trimer is encoded by a sequence selected from the group consisting of SEQ ID NO: 16, SEQ ID NO: 18, and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 16 and SEQ ID NO: 18. In some embodiments, the C3d trimer is encoded by at least one fragment of a nucleic acid sequence (e.g., a fragment having an antigenic sequence or at least one epitope) sequence is selected from the group consisting of SEQ ID NO: 16, SEQ ID NO: 18, and homologs having at least 90% (e.g., 90%, 91%, 92%, 93%, 94%, 95%, 96%, 97%, 98%, 99%, 99.8% or 99.9%) identity with a nucleic acid sequence selected from SEQ ID NO: 16 and SEQ ID NO: 18.

acccccgcag gctctgggga acagaacatg attggcatga caccaacagt cattgcggta cactacctgg
accagaccga acagtgggag aagttcggca tagagaagag gcaagaggcc ctggagctca tcaagaaagg gtacacccag
cagctggcct tcaaacagcc cagctctgcc tatgctgcct tcaacaaccg gccccccagc acctggctga cagcctacgt
ggtcaaggtc ttctctctag ctgccaacct catcgccatc gactctcacg tcctgtgtgg ggctgttaaa tggttgattc tggagaaaca
gaagccggat ggtgtctttc aggaggatgg gcccgtgatt caccaagaaa tgattggtgg cttccggaac gccaaggagg
cagatgtgtc actcacagcc ttcgtcctca tcgcactgca ggaagccagg gacatctgtg aggggcaggt caatagcctt
cctgggagca tcaacaaggc aggggagtat attgaagcca gttacatgaa cctgcagagg ccatacacag tggccattgc
tgggtatgcc ctggccctga tgaacaaact ggaggaacct tacctcggca agtttctgaa cacagccaaa gatcggaacc
gctgggagga gcctgaccag cagctctaca acgtagaggc cacatcctac gccctcctgg ccctgctgct gctgaaagac
tttgactctg tgccccctgt agtgcgctgg ctcaatgagc aaagatacta cggaggcggc tatggctcca cccaggctac
cttcatggta ttccaagcct tggcccaata tcaaacagat gtccctgacc ataaggactt gaacatggat gtgtccttcc
acctccccag cggctctggc gggggaggat cagggggtgg cggctctggc tcaacccccg caggctctgg ggaacagaac
atgattggca tgacaccaac agtcattgcg gtacactacc tggaccagac cgaacagtgg gagaagttcg gcatagagaa
gaggcaagag gccctggagc tcatcaagaa agggtacacc cagcagctgg ccttcaaaca gcccagctct gcctatgctg
ccttcaacaa ccggcccccc agcacctggc tgacagccta cgtggtcaag gtcttctctc tagctgccaa cctcatcgcc
atcgactctc acgtcctgtg tggggctgtt aaatggttga ttctggagaa acagaagccg gatggtgtct ttcaggagga
tgggcccgtg attcaccaag aaatgattgg tggcttccgg aacgccaagg aggcagatgt gtcactcaca gccttcgtcc
tcatcgcact gcaggaagcc agggacatct gtgaggggca ggtcaatagc cttcctggga gcatcaacaa ggcaggggag
tatattgaag ccagttacat gaacctgcag aggccataca cagtggccat tgctgggtat gccctggccc tgatgaacaa
actggaggaa ccttacctcg gcaagtttct gaacacagcc aaagatcgga accgctggga ggagcctgac cagcagctct
acaacgtaga ggccacatcc tacgccctcc tggccctgct gctgctgaaa gactttgact ctgtgccccc tgtagtgcgc
tggctcaatg agcaaagata ctacggaggc ggctatggct ccacccaggc taccttcatg gtattccaag ccttggccca
atatcaaaca gatgtccctg accataagga cttgaacatg gatgtgtcct tccacctccc cagcggctct ggcgggggag
gatcaggggg tggcggctct ggctcaaccc ccgcaggctc tggggaacag aacatgattg gcatgacacc aacagtcatt
gcggtacact acctggacca gaccgaacag tgggagaagt tcggcataga gaagaggcaa gaggccctgg agctcatcaa
gaaagggtac acccagcagc tggccttcaa acagcccagc tctgcctatg ctgccttcaa caaccggccc cccagcacct
ggctgacagc ctacgtggtc aaggtcttct ctctagctgc caacctcatc gccatcgact ctcacgtcct gtgtggggct
gttaaatggt tgattctgga gaaacagaag ccggatggtg tctttcagga ggatgggccc gtgattcacc aagaaatgat
tggtggcttc cggaacgcca aggaggcaga tgtgtcactc acagccttcg tcctcatcgc actgcaggaa gccagggaca
tctgtgaggg gcaggtcaat agccttcctg ggagcatcaa caaggcaggg gagtatattg aagccagtta catgaacctg
cagaggccat acacagtggc cattgctggg tatgccctgg ccctgatgaa caaactggag gaaccttacc tcggcaagtt
tctgaacaca gccaaagatc ggaaccgctg ggaggagcct gaccagcagc tctacaacgt agaggccaca tcctacgccc
tcctggccct gctgctgctg aaagactttg actctgtgcc ccctgtagtg cgctggctca atgagcaaag atactacgga
ggcggctatg gctccaccca ggctaccttc atggtattcc aagccttggc ccaatatcaa acagatgtcc ctgaccataa
ggacttgaac atggatgtgt ccttccacct ccccagc (SEQ ID NO: 16)

cacctcattg tgaccccctc gggctgcggg gaacagaaca tgatcggcat gacgcccacg gtcatcgctg
tgcattacct ggatgaaacg gagcagtggg agaagttcgg cctagagaag cggcaggggg ccttggagct catcaagaag
gggtacaccc agcagctggc cttcagacaa cccagctctg cctttgcggc cttcgtgaaa cgggcaccca gcacctggct
gaccgcctac gtggtcaagg tcttctctct ggctgtcaac ctcatcgcca tcgactccca agtcctctgc ggggctgtta
aatggctgat cctggagaag cagaagcccg acggggtctt ccaggaggat gcgcccgtga tacaccaaga aatgattggt
ggattacgga acaacaacga gaaagacatg gccctcacgg cctttgttct catctcgctg caggaggcta aagatatttg
cgaggagcag gtcaacagcc tgccaggcag catcactaaa gcaggagact tccttgaagc caactacatg aacctacaga
gatcctacac tgtggccatt gctggctatg ctctggccca gatgggcagg ctgaaggggc ctcttcttaa caaatttctg
accacagcca aagataagaa ccgctgggag gaccctggta agcagctcta caacgtggag gccacatcct atgccctctt
ggccctactg cagctaaaag actttgactt tgtgcctccc gtcgtgcgtt ggctcaatga acagagatac tacggtggtg
gctatggctc tacccaggcc accttcatgg tgttccaagc cttggctcaa taccaaaagg acgcccctga ccaccaggaa
ctgaaccttg atgtgtccct ccaactgccc agccgcggct ctggcggggg aggatcaggg ggtggcggct ctggctcaca
cctcattgtg accccctcgg gctgcgggga acagaacatg atcggcatga cgcccacggt catcgctgtg cattacctgg
atgaaacgga gcagtgggag aagttcggcc tagagaagcg gcagggggcc ttggagctca tcaagaaggg gtacacccag
cagctggcct tcagacaacc cagctctgcc tttgcggcct tcgtgaaacg ggcacccagc acctggctga ccgcctacgt
ggtcaaggtc ttctctctgg ctgtcaacct catcgccatc gactcccaag tcctctgcgg ggctgttaaa tggctgatcc
tggagaagca gaagcccgac ggggtcttcc aggaggatgc gcccgtgata caccaagaaa tgattggtgg attacggaac
aacaacgaga aagacatggc cctcacggcc tttgttctca tctcgctgca ggaggctaaa gatatttgcg aggagcaggt
caacagcctg ccaggcagca tcactaaagc aggagacttc cttgaagcca actacatgaa cctacagaga tcctacactg
tggccattgc tggctatgct ctggcccaga tgggcaggct gaaggggcct cttcttaaca aatttctgac cacagccaaa
gataagaacc gctgggagga ccctggtaag cagctctaca acgtggaggc cacatcctat gccctcttgg ccctactgca
gctaaaagac tttgactttg tgcctcccgt cgtgcgttgg ctcaatgaac agagatacta cggtggtggc tatggctcta
cccaggccac cttcatggtg ttccaagcct tggctcaata ccaaaaggac gcccctgacc accaggaact gaaccttgat
gtgtccctcc aactgcccag ccgcggctct ggcgggggag gatcaggggg tggcggctct ggctcacacc tcattgtgac
cccctcgggc tgcggggaac agaacatgat cggcatgacg cccacggtca tcgctgtgca ttacctggat gaaacggagc
agtgggagaa gttcggccta gagaagcggc agggggcctt ggagctcatc aagaaggggt acacccagca gctggccttc
agacaaccca gctctgcctt tgcggccttc gtgaaacggg cacccagcac ctggctgacc gcctacgtgg tcaaggtctt
ctctctggct gtcaacctca tcgccatcga ctcccaagtc ctctgcgggg ctgttaaatg gctgatcctg gagaagcaga
agcccgacgg ggtcttccag gaggatgcgc ccgtgataca ccaagaaatg attggtggat tacggaacaa caacgagaaa
gacatggccc tcacggcctt tgttctcatc tcgctgcagg aggctaaaga tatttgcgag gagcaggtca acagcctgcc
aggcagcatc actaaagcag gagacttcct tgaagccaac tacatgaacc tacagagatc ctacactgtg gccattgctg
gctatgctct ggcccagatg ggcaggctga aggggcctct tcttaacaaa tttctgacca cagccaaaga taagaaccgc
tgggaggacc ctggtaagca gctctacaac gtggaggcca catcctatgc cctcttggcc ctactgcagc taaaagactt
tgactttgtg cctcccgtcg tgcgttggct caatgaacag agatactacg gtggtggcta tggctctacc caggccacct
tcatggtgtt ccaagccttg gctcaatacc aaaaggacgc ccctgaccac (SEQ ID NO: 18)

In still further embodiments, the C3d trimer is at least about 70% identical to SEQ ID NO: 17 or SEQ ID NO: 19, for example, has at least about: 75%, 80%, 85%, 90%, 95%, 96%, 97%, 98%, 99% or 100% sequence identity to SEQ ID NO: 17 or SEQ ID NO: 19. In certain embodiments, the C3d trimer comprises an amino acid sequence that is about: 70%, 75%, 80%, 85%, 90%, 95%, 96%, 97%, 98%, 99% or 100% identical to SEQ ID NO: 17 or SEQ ID NO: 19. In some embodiments, the C3d trimer comprises an amino acid sequence having about 70%, 75%, 80%, 85%, 90%, 95%, 96%, 97%, 98%, 99% or 100% sequence identity to SEQ ID NO: 17 or SEQ ID NO: 19. In particular embodiments, the C3d trimer comprises a sequence having about 70-100% sequence identity to SEQ ID NO: 17 or SEQ ID NO: 19, for example, about: 75-100%, 75-99%, 80-100%, 80-98%, 85-100%, 85-97%, 90-100%, 90-96%, 95-100%, 96-100%, 97-100%, 98-100% or 99-100%.

TPAGSGEQNM IGMTPTVIAV HYLDQTEQWE KFGIEKRQEA LELIKKGYTQ
QLAFKQPSSA YAAFNNRPPS TWLTAYVVKV FSLAANLIAI DSHVLCGAVK
WLILEKQKPD GVFQEDGPVI HQEMIGGFRN AKEADVSLTA FVLIALQEAR
DICEGQVNSL PGSINKAGEY IEASYMNLQR PYTVAIAGYA LALMNKLEEP
YLGKFLNTAK DRNRWEEPDQ QLYNVEATSY ALLALLLLKD FDSVPPVVRW
LNEQRYYGGG YGSTQATFMV FQALAQYQTD VPDHKDLNMD VSFHLPSGSG
GGGSGGGGSG STPAGSGEQN MIGMTPTVIA VHYLDQTEQW EKFGIEKRQE
ALELIKKGYT QQLAFKQPSS AYAAFNNRPP STWLTAYVVK VFSLAANLIA
IDSHVLCGAV KWLILEKQKP DGVFQEDGPV IHQEMIGGFR NAKEADVSLT
AFVLIALQEA RDICEGQVNS LPGSINKAGE YIEASYMNLQ RPYTVAIAGY
ALALMNKLEE PYLGKFLNTA KDRNRWEEPD QQLYNVEATS YALLALLLLK
DFDSVPPVVR WLNEQRYYGG GYGSTQATFM VFQALAQYQT DVPDHKDLNM
DVSFHLPSGS GGGGSGGGGS GSTPAGSGEQ NMIGMTPTVI AVHYLDQTEQ
WEKFGIEKRQ EALELIKKGY TQQLAFKQPS SAYAAFNNRP PSTWLTAYVV
KVFSLAANLI AIDSHVLCGA VKWLILEKQK PDGVFQEDGP VIHQEMIGGF
RNAKEADVSL TAFVLIALQE ARDICEGQVN SLPGSINKAG EYIEASYMNL
QRPYTVAIAG YALALMNKLE EPYLGKFLNT AKDRNRWEEP DQQLYNVEAT
SYALLALLLL KDFDSVPPVV RWLNEQRYYG GGYGSTQATF MVFQALAQYQ
TDVPDHKDLN MDVSFHLPS (SEQ ID NO: 17)

HLIVTPSGCG EQNMIGMTPT VIAVHYLDET EQWEKFGLEK RQGALELIKK
GYTQQLAFRQ PSSAFAAFVK RAPSTWLTAY VVKVFSLAVN LIAIDSQVLC
GAVKWLILEK QKPDGVFQED APVIHQEMIG GLRNNNEKDM ALTAFVLISL
QEAKDICEEQ VNSLPGSITK AGDFLEANYM NLQRSYTVAI AGYALAQMGR
LKGPLLNKFL TTAKDKNRWE DPGKQLYNVE ATSYALLALL QLKDFDFVPP
VVRWLNEQRY YGGGYGSTQA TFMVFQALAQ YQKDAPDHQE LNLDVSLQLP
SRGSGGGGSG GGGSGSHLIV TPSGCGEQNM IGMTPTVIAV HYLDETEQWE
KFGLEKRQGA LELIKKGYTQ QLAFRQPSSA FAAFVKRAPS TWLTAYVVKV
FSLAVNLIAI DSQVLCGAVK WLILEKQKPD GVFQEDAPVI HQEMIGGLRN
NNEKDMALTA FVLISLQEAK DICEEQVNSL PGSITKAGDF LEANYMNLQR
SYTVAIAGYA LAQMGRLKGP LLNKFLTTAK DKNRWEDPGK QLYNVEATSY
ALLALLQLKD FDFVPPVVRW LNEQRYYGGG YGSTQATFMV FQALAQYQKD
APDHQELNLD VSLQLPSRGS GGGGSGGGGS GSHLIVTPSG CGEQNMIGMT
PTVIAVHYLD ETEQWEKFGL EKRQGALELI KKGYTQQLAF RQPSSAFAAF
VKRAPSTWLT AYVVKVFSLA VNLIAIDSQV LCGAVKWLIL EKQKPDGVFQ
EDAPVIHQEM IGGLRNNNEK DMALTAFVLI SLQEAKDICE EQVNSLPGSI
TKAGDFLEAN YMNLQRSYTV AIAGYALAQM GRLKGPLLNK FLTTAKDKNR
WEDPGKQLYN VEATSYALLA LLQLKDFDFV PPVVRWLNEQ RYYGGGYGST
QATFMVFQAL AQYQKDAPDH QELNLDVSLQ LPSR (SEQ ID NO: 19)

### Methods of Treatment

In some embodiments, the disclosure provides for methods of treatment and methods of enhancing efficacy and/or potency of treatment comprising administration of the compositions, constructs and nanoparticles described herein.

As used herein, “therapy,” “treat,” “treating,” or “treatment” means inhibiting or relieving a condition in a subject in need thereof. For example, a therapy or treatment refers to any of: (i) the prevention of symptoms associated with a disease or disorder; (ii) the postponement of development of the symptoms associated with a disease or disorder; and/or (iii) the reduction in the severity of such symptoms that will, or are expected, to develop with said disease or disorder. The terms include ameliorating or managing existing symptoms, preventing additional symptoms, and ameliorating or preventing the underlying causes of such symptoms. Thus, the terms denote that a beneficial result is being conferred on at least some of the subjects (e.g., humans) being treated. Many therapies or treatments are effective for some, but not all, subjects that undergo the therapy or treatment.

As used herein, the term “effective amount” means an amount of a composition, that when administered alone or in combination to a cell, tissue, or subject, is effective to achieve the desired therapy or treatment under the conditions of administration. For example, an effective amount is one that would be sufficient to produce an immune response to bring about effectiveness of a therapy or treatment. The effectiveness of a therapy or treatment (e.g., eliciting a humoral and/or cellular immune response) can be determined by suitable methods known in the art.

In some embodiments the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years.

In some embodiments, the present disclosure provides for a method of treatment for an infectious disease. In some embodiments, the infectious disease is a coronavirus, influenza virus, respiratory syncytial virus (RSFV), human immunodeficiency virus, zika virus, Epstein-Barr virus, herpes simplex virus, rabies, cytomegalovirus, mycobacterium tuberculosis, or a combination thereof. In still further embodiments, the infectious disease is a SARS-CoV-2 or SARS-CoV-2-like virus. In some embodiments, the disclosure provides for an infection disease agent, wherein the agent is a spike protein (SP), a receptor binding domain (RBD), or a combination thereof.

In some embodiments, the spike protein is at least about 70% identical to SEQ ID NO: 21, for example, has at least about: 75%, 80%, 85%, 90%, 95%, 96%, 97%, 98%, 99% or 100% sequence identity to SEQ ID NO: 21. In certain embodiments, the spike protein comprises an amino acid sequence that is about: 70%, 75%, 80%, 85%, 90%, 95%, 96%, 97%, 98%, 99% or 100% identical to SEQ ID NO: 21. In some embodiments, the spike protein comprises an amino acid sequence having about 70%, 75%, 80%, 85%, 90%, 95%, 96%, 97%, 98%, 99% or 100% sequence identity to SEQ ID NO: 21. In particular embodiments, the spike protein comprises a sequence having about 70-100% sequence identity to SEQ ID NO: 21, for example, about: 75-100%, 75-99%, 80-100%, 80-98%, 85-100%, 85-97%, 90-100%, 90-96%, 95-100%, 96-100%, 97-100%, 98-100% or 99-100%.

In still further embodiments, the spike protein is encoded by a polynucleotide sequence. In some embodiments, the polynucleotide is at least about 70% identical to SEQ ID NO: 20, for example, has at least about: 75%, 80%, 85%, 90%, 95%, 96%, 97%, 98%, 99% or 100% sequence identity to SEQ ID NO: 20. In certain embodiments, the polynucleotide comprises a nucleotide sequence that is about: 70%, 75%, 80%, 85%, 90%, 95%, 96%, 97%, 98%, 99% or 100% identical to SEQ ID NO: 20. In some embodiments, the polynucleotide comprises a nucleotide sequence having about 70%, 75%, 80%, 85%, 90%, 95%, 96%, 97%, 98%, 99% or 100% sequence identity to SEQ ID NO: 20. In particular embodiments, the polynucleotide comprises a nucleotide sequence having about 70-100% sequence identity to SEQ ID NO: 20, for example, about: 75-100%, 75-99%, 80-100%, 80-98%, 85-100%, 85-97%, 90-100%, 90-96%, 95-100%, 96-100%, 97-100%, 98-100% or 99-100%.

In some embodiments, the receptor binding domain is at least about 70% identical to SEQ ID NO: 23 or SEQ ID NO: 25, for example, has at least about: 75%, 80%, 85%, 90%, 95%, 96%, 97%, 98%, 99% or 100% sequence identity to SEQ ID NO: 23 or SEQ ID NO: 25. In certain embodiments, the receptor binding domain comprises an amino acid sequence that is about: 70%, 75%, 80%, 85%, 90%, 95%, 96%, 97%, 98%, 99% or 100% identical to SEQ ID NO: 23 or SEQ ID NO: 25. In some embodiments, the receptor binding domain comprises an amino acid sequence having about 70%, 75%, 80%, 85%, 90%, 95%, 96%, 97%, 98%, 99% or 100% sequence identity to SEQ ID NO: 23 or SEQ ID NO: 25. In particular embodiments, the receptor binding domain comprises a sequence having about 70-100% sequence identity to SEQ ID NO: 23 or SEQ ID NO: 25, for example, about: 75-100%, 75-99%, 80-100%, 80-98%, 85-100%, 85-97%, 90-100%, 90-96%, 95-100%, 96-100%, 97-100%, 98-100% or 99-100%.

In still further embodiments, the receptor binding domain is encoded by a polynucleotide sequence. In some embodiments, the polynucleotide is at least about 70% identical to SEQ ID NO: 22 or SEQ ID NO: 24, for example, has at least about: 75%, 80%, 85%, 90%, 95%, 96%, 97%, 98%, 99% or 100% sequence identity to SEQ ID NO: 22 or SEQ ID NO: 24. In certain embodiments, the polynucleotide comprises a nucleotide sequence that is about: 70%, 75%, 80%, 85%, 90%, 95%, 96%, 97%, 98%, 99% or 100% identical to SEQ ID NO: 22 or SEQ ID NO: 24. In some embodiments, the polynucleotide comprises a nucleotide sequence having about 70%, 75%, 80%, 85%, 90%, 95%, 96%, 97%, 98%, 99% or 100% sequence identity to SEQ ID NO: 22 or SEQ ID NO: 24. In particular embodiments, the polynucleotide comprises a nucleotide sequence having about 70-100% sequence identity to SEQ ID NO: 22 or SEQ ID NO: 24, for example, about: 75-100%, 75-99%, 80-100%, 80-98%, 85-100%, 85-97%, 90-100%, 90-96%, 95-100%, 96-100%, 97-100%, 98-100% or 99-100%.

In still other aspects, the disclosure provides for a method of treating cancer. In certain embodiments, the cancer is melanoma, colorectal cancer, high-risk melanoma, human papilloma virus, head and neck squamous carcinoma, non-small cell lung cancer, New York esophageal squamous cell carcinoma, or a combination thereof.

In some embodiments, the agent is a HPV16-derived tumor antigen, E6 viral oncoprotein, E7 viral oncoprotein, melanoma-associated antigen, mucin1, or trophoblast glycoprotein.

In still further embodiments, the method comprises administering to the subject an effective amount of the composition, or a pharmaceutically acceptable salt thereof.

The term “pharmaceutically acceptable salts” embraces salts commonly used to form alkali metal salts and to form addition salts of free acids or free bases. The nature of the salt is not critical, provided that it is pharmaceutically acceptable.

Suitable pharmaceutically acceptable acid addition salts may be prepared from an inorganic acid or an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. Pharmaceutically acceptable basic/cationic salts also include, the diethanolamine, ammonium, ethanolamine, piperazine and triethanolamine salts.

All of these salts may be prepared by conventional means by treating, for example, a composition described herein with an appropriate acid or base.

In some embodiments, compositions of the disclosure are administered in a delivery vehicle comprising a nanocarrier selected from the group consisting of a lipid, a polymer and a lipo-polymeric hybrid. In still further embodiments, the first and second polynucleotides are encapsulated in a liposomal composition, lipid nanoparticle, polymer nanoparticle, virus-like particle, nanowire, exosome, hybrid lipid/polymer nanoparticle, core-shell nanoparticle, nanoparticle mimic, and/or combinations thereof. In some embodiments, the first and second polynucleotides are encapsulated in the same nanocarrier. In still further embodiments, the first and second polynucleotides are encapsulated in different nanocarriers. In some embodiments, the lipid nanoparticle is ionizable. In some embodiments, the lipid nanoparticle is partly (partially) ionizable. In some embodiments, the lipid nanoparticle is fully ionizable. In some embodiments, the lipid nanoparticle is at least about: 10%, 15%, 20%, 25%, 30%, 35%, 40%, 45%, 50%, 55%, 60%, 65%, 70%, 75%, 80%, 85%, 90%, 95% or 98% ionizable. In some embodiments, the lipid nanoparticle is about: 10%, 15%, 20%, 25%, 30%, 35%, 40%, 45%, 50%, 55%, 60%, 65%, 70%, 75%, 80%, 85%, 90%, 95%, 98% or 100% ionizable.

As used herein, the term “reducing” or “reduce” refers to modulation that decreases risk (e.g., the level prior to or in an absence of modulation by the agent). In some embodiments, the agent (e.g., composition) reduces risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In certain embodiments, the agent (e.g., composition) decreases risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference. In particular embodiments, the agent (e.g., composition) decreases risk, by at least about 5% relative to the reference, e.g., by at least about: 10%, 15%, 20%, 25%, 30%, 35%, 40%, 45%, 50%, 55%, 60%, 65%, 70%, 75%, 80%, 85%, 90%, 95% or 98% relative to the reference.

As used herein, “exosome” means a membrane-bounded sub-cellular structure which may comprise proteins, messenger ribonucleic acids (mRNA), and other biologically active substances. In certain embodiments, the exosome is that of a macrophage. In some embodiments, the macrophage exosome is determined in tissue sample, cell, or serum sample. In some embodiments, the proteins, messenger ribonucleic acids, and other biologically active substances may be freely released. Further description of exosomes suitable for use in the present disclosure are described in Aslan, C., Kiaie, S.H., Zolbanin, N.M. et al. Exosomes for mRNA delivery: a novel biotherapeutic strategy with hurdles and hope. BMC Biotechnol 21, 20 (2021) (incorporated in its entirety herein by reference).

In some embodiments, the RNA is unmodified. In some embodiments, the RNA can be chemically modified, for example to improve its properties, e.g, used to improve the properties and efficacy of the RNA. A number of chemical modification have been developed to improve the in vivo properties of nucleic acids. Chemical modifications can be used alone or in combination and the number of modified nucleotides can vary relative to the number that remain as unmodified RNA. Chemical modification can also improve the in vivo properties of nucleic acids. Each can be used alone or in combination, and the number of modified nucleotides can vary relative to the number that remain as unmodified RNA. Some modifications are introduced at most or all bases of both RNA strands, whereas other modifications are placed at certain positions.

### Compositions

In some embodiments, the disclosure provides for a composition that is a pharmaceutically acceptable composition.

As used herein, the term “pharmaceutically acceptable” refers to species which are, within the scope of sound medical judgment, suitable for use without undue toxicity, irritation, allergic response and the like, and are commensurate with a reasonable benefit/risk ratio. For example, a substance is pharmaceutically acceptable when it is suitable for use in contact with cells, tissues or organs of animals or humans without excessive toxicity, irritation, allergic response, immunogenicity or other adverse reactions, in the amount used in the dosage form according to the dosing schedule, and commensurate with a reasonable benefit/risk ratio.

A desired dose may conveniently be administered in a single dose, for example, such that the agent is administered once per day, or as multiple doses administered at appropriate intervals, for example, such that the agent is administered 2, 3, 4, 5, 6 or more times per day. The daily dose can be divided, especially when relatively large amounts are administered, or as deemed appropriate, into several, for example 2, 3, 4, 5, 6 or more, administrations. Typically, the compositions will be administered from about 1 to about 6 (e.g., 1, 2, 3, 4, 5 or 6) times per day or, alternatively, as an infusion (e.g., a continuous infusion).

Determining the dosage and route of administration for a particular agent, patient and disease or condition is well within the abilities of one of skill in the art. Preferably, the dosage does not cause or produces minimal adverse side effects.

Doses lower or higher than those recited above may be required. Specific dosage and treatment regimens for any particular subject will depend upon a variety of factors, for example, the activity of the specific agent employed, the age, body weight, general health status, sex, diet, time of administration, rate of excretion, drug combination, the severity and course of the disease, condition or symptoms, the subject’s disposition to the disease, condition or symptoms, the judgment of the treating physician and the severity of the particular disease being treated. The amount of an agent in a composition will also depend upon the particular agent in the composition.

In some embodiments, the concentration of one or more active agents provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, or 0.01% w/w, w/v or v/v; and/or greater than 90%, 80%, 70%, 60%, 50%, 40%, 30%, 20%, 10%, 5%, 1%, 0.5%, 0.4%, 0.3%, 0.2%, 0.1%, or 0.01% w/w, w/v, or v/v.

In some embodiments, the concentration of one or more active agents provided in a composition is in the range from about 0.01% to about 50%, about 0.01% to about 40%, about 0.01% to about 30%, about 0.05% to about 25%, about 0.1% to about 20%, about 0.15% to about 15%, or about 1% to about 10% w/w, w/v or v/v. In some embodiments, the concentration of one or more active agents provided in a composition is in the range from about 0.001% to about 10%, about 0.01% to about 5%, about 0.05% to about 2.5%, or about 0.1% to about 1% w/w, w/v or v/v.

In certain embodiments, the administration of the composition may be carried out in any manner, e.g., by parenteral or nonparenteral administration, including by aerosol inhalation, injection, infusions, ingestion, transfusion, implantation or transplantation. For example, the compositions described herein may be administered to a patient trans-arterially, intradermally, subcutaneously, intratumorally, intramedullary, intranodally, intramuscularly, by intravenous (i.v.) injection, intranasally, intrathecally or intraperitoneally. In one aspect, the compositions of the present disclosure are administered intravenously. In one aspect, the compositions of the present disclosure are administered to a subject by intramuscular or subcutaneous injection. The compositions may be injected, for instance, directly into a tumor, lymph node, tissue, organ, or site of infection.

In some embodiments, compositions as described herein are used in combination with other known agents and therapies. Administered “in combination”, as used herein, means that two (or more) different treatments are delivered to the subject during the course of the subject’s treatment e.g., the two or more treatments are delivered after the subject has been diagnosed with the disease and before the disease has been cured or eliminated or treatment has ceased for other reasons. In some embodiments, different treatments (e.g., additional therapeutics) can be administered simultaneously or sequentially.

In some embodiments, the mRNA is circular. In some embodiments the first polynucleotide sequence is circular. In some embodiments the second polynucleotide sequence in circular. In still further embodiments, the first polynucleotide sequence operably connected to the second polynucleotide sequence comprise a circular RNA.

In some embodiments, the composition comprises a promoter. In still further embodiments, the promoter is a selective promoter. In some embodiments of the disclosure, the selective promoter is CD11b. In one embodiment, the vector further comprises an RNA polymerase promoter. In another embodiment, the RNA polymerase promoter is a T7 virus RNA polymerase promoter, T6 virus RNA polymerase promoter, SP6 virus RNA polymerase promoter, T3 virus RNA polymerase promoter, or T4 virus RNA polymerase promoter. In some embodiments, the construct is enclosed in a nanoparticle. As used herein, a “nanoparticle” or “nanocarrier” is used to mean encapsulation in a liposomal composition, lipid nanoparticle, polymer nanoparticle, virus-like particle, nanowire, exome, hybrid lipid/polymer nanoparticle, core-shell nanoparticle, nanoparticle mimic, and/or combinations thereof. In some embodiments, the construct is enclosed in an LNP. In one embodiment, a four-composition formulation ratio was followed to prepare the LNPs, which contains one or more lipids (e.g., ionizable lipids). In some embodiments, the LNP comprises an ionizable lipid, 2-dioleoyl-sn-glycerol-3-phosphoethanolamine (DOPE), as a helper lipid, cholesterol, and 1,2-dimyristoyl-sn-glycerol-3-phosphoethanolamine-N-[methoxy-(polyethyleneglycol)-2000](ammonium salt)(C14-PEG). In some embodiments, the construct or constructs is enclosed in a liposome. As used herein, the term “liposome” means a lamellar, multilamellar, or solid lipid nanoparticle vesicle. In some embodiments, a liposome as used herein can be formed by mixing one or more lipids or by mixing one or more lipids and polymer(s). Thus, as used herein, the term “liposome” includes lipid- and polymer-based nanoparticles.

### EXEMPLIFICATION

### Methods

### Plasmid and mRNA Synthesis

mRNA was transcribed through in vitro transcription (IVT) from linearized plasmids containing a T7 promoter upstream of the relevant CDS flanked by partial cytomegalovirus (CMV) 5′ UTR and a partial human growth hormone (hGH) 3′ UTR. In short, an IVT template cloning vector was generated by cloning a cassette containing the T7 promoter sequence, CMV 5′ UTR, CDS cloning site flanked by two BsaI sites, and hGH 3′ UTR into the pUC19 vector. For generating specific IVT templates, the desired CDS flanked by two BsaI sites was synthesized as a block (IDT) and cloned into the template using the BsaI-HF v2 Golden Gate Assembly Kit (New England Biolabs). CDSs comprised either the full-length wild-type spike protein, wild-type receptor binding domain, or the Delta variant (B.1.617.2) receptor-binding domain of SARS-CoV-2 alone or fused to murine C3d₃ previously described in Dempsey, P.W., Allison, M.E.D., Akkaraju, S., Goodnow, C.C. & Fearon, D.T. C3d of complement as a molecular adjuvant: Bridging innate and acquired immunity. Science 271, 348-350 (1996) (incorporated herein by reference). IVT templates were then linearized using EcoRI, and mRNA was transcribed using the HiScribe T7 High Yield RNA Synthesis Kit (New England Biolabs). Capping and tailing were performed post-transcriptionally using the Vaccinia Capping System and E. coli Poly(A) Polymerase (New England Biolabs). The resulting capped and tailed mRNA was purified using the Monarch RNA Cleanup Kit (New England Biolabs). Purified mRNA was analyzed by gel electrophoresis to confirm the size and ensure purity.

### LNP Formulation

LNPs were synthesized by mixing an aqueous phase containing the mRNA with an ethanol phase containing the lipids in a microfluidic chip device. The ethanol phase was prepared by solubilizing a mixture of ionizable lipid, helper phospholipid, cholesterol (Chol, Sigma-Aldrich), PEG-lipid, and in some instances, sodium lauryl sulfate (SLS, Sigma-Aldrich) at predetermined molar ratios. All helper phospholipids and PEG-lipids were purchased from Avanti. For LPS-containing formulations, LPS was dissolved in ethanol at 10 mg/mL and added to the ethanol phase. The aqueous phase was prepared in a 10 mM citrate buffer with corresponding mRNA (Firefly luciferase, OVA, SARS-CoV-2 constructs). The aqueous and ethanol phases were mixed in a microfluidic device at a 3:1 ratio by syringe pumps to a final mRNA concentration of 0.1 mg/mL. The resultant formulation was dialyzed against PBS overnight in a 20 K MWCO dialysis cassette (ThermoFisher) at 4° C.

### LNP Characterization

The diameter of the LNPs was measured using dynamic light scattering (Zetasizer, Malvern). LNP diameters are reported as the largest intensity mean peak average, constituting>95% of the nanoparticles in the sample. mRNA encapsulation efficiencies were measured by a modified Quanti-iT Ribogreen RNA assay (Invitrogen) as previously described.

### Bioluminescence

To evaluate the transfection efficiency of mFFL LNPs, the in vitro bioluminescence was measured using ONE-Glo Luciferase Assay System (Promega) according to the manufacturer’s instructions. The bioluminescence signal in cells was quantified using the Tecan Infinite M200 Pro plate reader (Tecan). For measuring in vivo bioluminescence, 6 h after the injection of mRNA LNPs, mice were injected intraperitoneally with 0.2 ml XenoLight D-luciferin (10 mg/mL in DPBS, PerkinElmer). Mice were anesthetized in a ventilated anesthesia chamber with 2.5% isofluorane in oxygen and imaged 10 min after luciferin injection with an in vivo imaging system (IVIS, PerkinElmer). Luminescence was quantified using the Living Image software (PerkinElmer).

### Western Blot and ELISA of SARS-Co V-2 Antigens With or Without C3d

HEK293T cells were plated at 5×10⁴ cells/well in a 24-well plate and grown overnight. Cells were then transfected with 1 µg of mRNA encoding for SARS-CoV2 antigens with or without C3d using MessengerMax (ThermoFisher) according to manufacturer’s instructions. Cells were lysed with RIPA buffer 24 h after transfection and subsequently used for Western blot analysis or ELISA. For western blot analysis, following lysis, total protein concentration of samples was determined via Bicinchoninic Acid (BCA) assay (Thermo Scientific #23227). For each sample, 15 ug total protein was added to 20 µl 2x Sample Buffer (Bio Rad #1610738) and 4 µl 10x NuPage reducing agent (Invitrogen #NP0004) and the total volume fixed to 41 µl with water as necessary. Solutions were then boiled at 95° C. for 5 minutes to fully denature all proteins.

Samples were loaded into the wells of either a 4-12% or 7.5% Tris-Glycine gel (Invitrogen XP04122 or Bio Rad #5671025) with a protein ladder (Bio Rad #1610377 or Licor #928-60000) and Tris-Glycine SDS running buffer, and the gel was run for 1.5 hours at 120 V (4-12% gel) or 30 minutes (7.5% gel).

Gels for western blotting were then dry-transferred to a nitrocellulose membrane (Invitrogen IB21001 and IB23002) on an iBlot2 using Preset 0 (1 min 20 V, 4 min 23 V, 2 min 25 V) according to the manufacturer’s instructions. The membrane was then blocked in 5% BSA in Tris-Buffered Saline with Tween (TBST) or Licor Intercept TBS Blocking Buffer (Licor #927-60001) for 1 hour at room temperature. Primary antibodies against β-Actin (Cell Signaling Technology 3700S or 4970), SARS-CoV-2 Spike Protein RBD or S2 (Sino Biological 40592-T62 and 40590-T62), and C3d (R&D Systems AF2655) were prepared in blocking buffer at concentrations of 1:5000, 1:1500 and 1:1500 respectively, and a primary incubation step was carried out overnight at 4° C. or for 2 hours at room temperature. The membrane was washed 4x with TBST.

For chemiluminescent blots, the membrane was then incubated with chemiluminescent secondary antibodies against goat (Invitrogen 31402), mouse (Jackson Immunoresearch Labs 315-035-048), and rabbit (Cell Signaling Technology 7074S) IgG for 1 hour. The membrane was then washed 5x with TBST, soaked in chemiluminescent substrate (Thermo Scientific 32106) and imaged using a Bio Rad Gel Doc imager.

For fluorescent blots, the membrane was then incubated with fluorescent secondary antibodies against goat (Licor #926-68074) and rabbit (Licor 926-32213) IgG for 1 hour. The membrane was then washed 4x with TBST and 1x with TBS and imaged using a Licor Odyssey imager.

ELISA analysis of SARS-CoV-2 antigens was performed with a commercial SARS-CoV-2 (2019-nCoV) Spike Detection ELISA kit (Sino Biological KIT40591) / RBD detection ELISA kit (Sino Biological KIT40592) according to the manufacturer’s instructions. The assay is based on a double-antibody sandwich principle that detects SARS-CoV-2 Spike or RBD protein in samples. Briefly, a monoclonal antibody specific for SARS-CoV-2 Spike or RBD protein was pre-coated onto plate wells. Standards and cell lysates were added to the wells and incubated for 2 h at room temperature. After three washes, plates were incubated with HRP-conjugated another anti-Spike or anti-RBD antibody for 1 h at room temperature, followed by three washes and incubation with TMB substrate. The absorbance at 450 nm was measured. A standard curve of absorbance at 450 nm versus concentration was fit with a linear equation for accurate Spike or RBD quantification.

### Animal Experiments

All procedures were performed under an animal protocol approved by the Massachusetts Institute of Technology Committee on Animal Care (CAC) and the guidelines for animal care in an MIT animal facility. C57BL/6J, 6-8 weeks of age, were purchased from Jackson Laboratories and housed in an MIT animal facility.

### Mouse Immunization

Mice were anesthetized in a ventilated anesthesia chamber with 2.5% isofluorane in oxygen. LNPs (0.05 mL volume per mouse at specified doses) were injected intramuscularly into the hind leg of mice. Mice were put back in their cages and monitored for signs of distress and local inflammation at the injection site. Blood was drawn from mice by either tail vein or cardiac puncture into serum separation tubes at different time points. The serum was isolated by centrifugation to characterize antigen-specific antibodies or systemic cytokine levels.

### ELISA of Mouse Serum

100 µL antigen solution (1 µg/mL of either wild-type Spike protein, wild-type RBD, or RBD Delta B.1.617.2 variant protein, Leinco Technologies) prepared in 0.1 M sodium carbonate buffer, pH 10.5, was used to coat every well of a flat-bottomed, high-binding 96-well plate (Grenier). Plates were incubated at 4° C. overnight. After removing antigen solutions, the plates were washed five times using PBST (0.05% v/v Tween 20; PBS 7.4) and then filled with blocking buffer at 37° C. (1% BSA solution in 0.1 M Tris buffer, pH 8.0). After incubation at room temperature for one h, the blocking buffer was removed, and all wells were washed by PBST another five times. Two-fold serial dilutions of mouse sera in PBS containing 1% BSA were added to the plates (100 µL/well) and incubated for one h at 37° C. The plates were then washed five times with PBST. Goat anti-mouse IgG conjugated to HRP (Cell Signaling Technology, dissolved 1:2000) in PBS with 1% BSA was used as the secondary antibody to detect IgG. After adding the secondary antibody solution, plates were incubated at 37° C. for one h and then washed five times using PBST before the addition of 50 µL/well HRP substrate 3,3′,5,5′-tetramethylbenzidine (Cell Signaling Technology). The plates were shaken for 15 min, and a 50 µL stop solution (0.2 M H2SO4) was added to each well. Absorbance at 450 (signal) and 570 nm (background) was recorded by a Tecan microplate reader. End-point titers were defined as the highest serum dilution that gave an optical density difference above 0.05 compared to serum from sham vaccinated mice at the same dilution.

### ELISpot Assay

The IFN- T cell response was assessed using the Mouse IFN-gamma ELISpot Kit (R&D Systems), following the manufacturer’s instructions. Briefly, anti-IFN- pre-coated plates were blocked with DMEM + 10% FBS for at least 30 min. Splenocytes were added at 2.5 × 10⁵ cells per well for negative control (media only), and SIINFEKL peptide or SARS-CoV-2 peptide pools (15-mers overlapping by 11; JPT Peptides) (1 µg mL⁻¹) in 200 µL final volume per well. Plates were incubated overnight at 5% CO2 in a 37° C. incubator and developed per the manufacturer’s protocol. Once dried, plates were read using CTL ImmunoSpot Series S five Versa ELISpot Analyzer (S5Versa-02-9038) and analyzed by ImmunoCapture v.6.3 software.

### Flow Cytometry Analyses for Mouse Splenocytes

10⁶ mouse splenocytes were stimulated with 2 µg/ml SARS-CoV-2 peptide pools (15-mers overlapping by 11; JPT Peptides) for 2 hours at 37° C. with 5% CO2. Following two washes with PBS, splenocytes cells were pre-incubated with anti-CD16/32 antibodies and stained on ice with fluorophore-labeled antibodies against B220, CD21, CD4, CD44, and CD62L. Flow data were evaluated using a BD LSR II flow cytometer and analyzed using FlowJo software.

### Serum Cytokine and Chemokine Analysis

Systemic cytokines and chemokines in the serum were measured over time by bead-based Bio-Plex Pro Mouse Cytokine Assay. Briefly, mouse sera (50 µL) were incubated with antibody-conjugated magnetic beads for 30 min in the dark. After washing, the detection antibody was added to the wells and incubated in the dark for 30 min under continuous shaking (300 rpm). After washing three times, streptavidin-phycoerythrin was added to each well and incubated while protected from light for 10 min under the same shaking conditions. Finally, after three-time washings and re-suspension in the assay buffer and shaking, the expression of the mouse cytokines and chemokines was measured immediately using Bioplex 200 system with HTF and Pro II Wash station, and the data were analyzed using the Bioplex Data Pro software (Bio-Rad Laboratories, Inc., Hercules, CA).

### Antigen-specific Isotype Titer and FcR-Binding

To determine isotype titer and FcR-binding, a multiplex Luminex assay was performed. Antigen was covalently linked to carboxyl-modified Magplex© Luminex beads using sulfo-NHS (Thermo Fisher) and ethyl dimethyl aminopropyl carbodiimide hydrochloride EDC (Thermo Fisher) to form ester-NHS linkages. To form immune complexes, serum was diluted (1:100 for IgG, IgG1, IgG2b, IgG2c, IgG3, IgM, and IgA and 1:500 for all FcRs), and diluted serum and antigen-coupled microspheres were mixed in 384-well plates. Plates were incubated overnight at 4° C., shaking at 700 rpm. Immune complexes were washed (1x PBS with 0.1% BSA 0.02% Tween-20). To detect antigen-specific titer, PE-coupled goat anti-mouse IgG, IgG1, IgG2b, IgG2c, IgG3, IgM, or IgA (Southern Biotech) was added to plates. To detect antigen-specific FcR-binding, Avi-tagged FcRs (Duke Human Vaccine Institute) was biotinylated using a BirA500 kit (Avidity) per manufacturer instructions. Biotinylated FcRs were fluorescently labeled using streptavidin-PE (Agilent), and FcR-PE was added to immune complex plates. Fluorescence was determined using an iQue (Intellicyt). The assay was run in duplicate, and the data reported shows the average of the replicates. The data represents the median fluorescence intensity (MFI).

### Data Analysis

Principal component analysis (PCA) and polar plots were generated in Python (version 3.7). Before analysis, data were log10-transformed and centered, and scaled. For building polar plots, data was percentile ranked. Polar plots showed the mean percentile rank for each feature in a group and were visualized using Plotly. PCA was performed using sklearn, decomposition module, and visualized using matplotlib.

### Example 1. Construction of C3dfusion mRNA

In one aspect, the present disclosure provides for an adjuvating mRNA vaccine that is effective at low dosage, by integrating a molecular adjuvant, C3d, into an mRNA vaccine sequence, e.g., to provide for an immunostimulatory effect that is synergistic with the cyclic lipid shells.

A gene fragment encoding three copies of murine C3d, amplified by PCR, was Golden gate cloned into a cloning plasmid. In an embodiment of the disclosure the cloning plasmid further comprises a full-length SARS-CoV-2 spike protein (SP). In another embodiment, the cloning plasmid further comprises a SARS-CoV-2 receptor-binding domain (RBD). In yet another embodiment, the cloning plasmid further comprises a fragment of the SP responsible for viral entry.

mRNA sequences encoding SP, RBD, SP-C3d and RBD-C3d were prepared by in vitro transcription (IVT), followed by enzymatic 5′ capping and poly-A tailing (FIG. 1A).

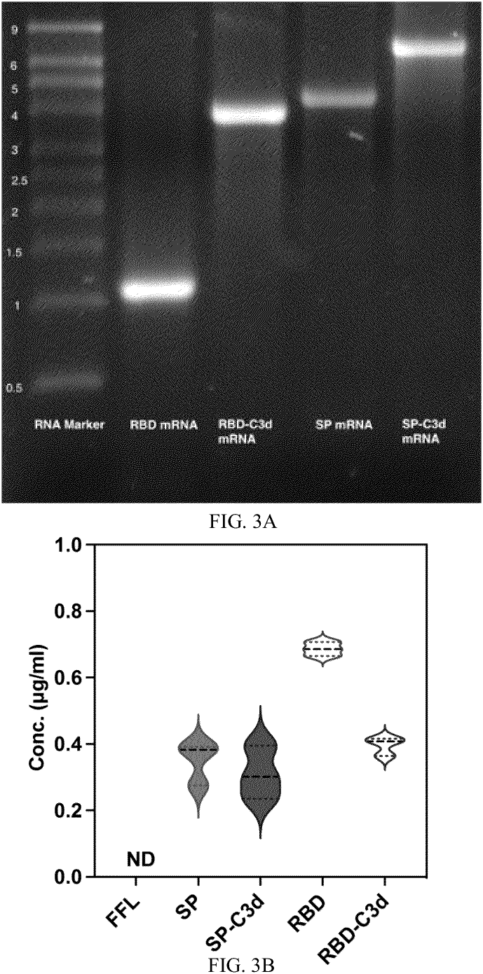

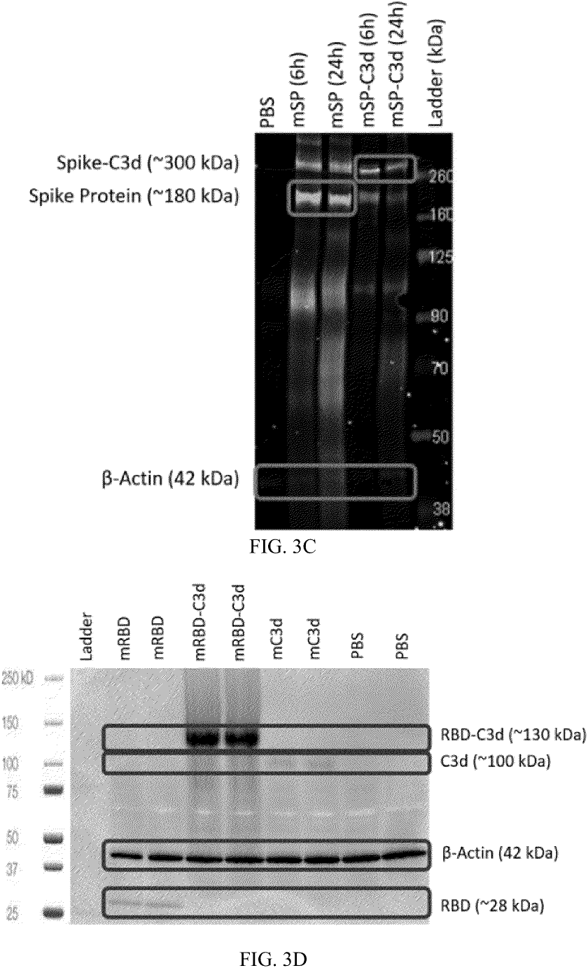

Results of gel electrophoresis indicate that the estimated sizes of mRNA encoding RBD, RBD-C3d, SP and SP-C3d are 0.9 kilobases (kb), 3.7 kb, 4.1 kb and 6.8 kb respectively (FIG. 3A). The as-prepared IVT mRNA was then formulated with ionizable lipid cKK-E12 nanoparticles (LNPs) using a microfluidic device (described in Kauffman, K.J. et al. Optimization of Lipid Nanoparticle Formulations for mRNA Delivery in Vivo with Fractional Factorial and Definitive Screening Designs. Nano Lett 15, 7300-7306 (2015), incorporated herein by reference).

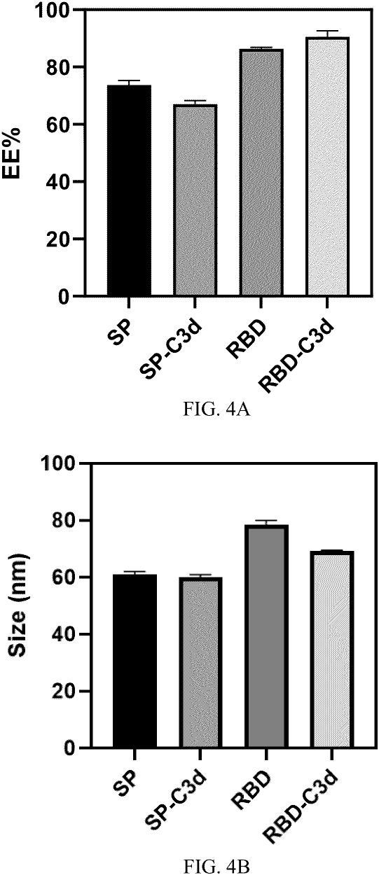

To confirm the efficiency of LNPs in transfection, each mRNA LNP was incubated with HEK293T cells and then the expression of protein antigens was analyzed. High expression of SARS-CoV-2 SP, SP-C3d, RBD, and RBD-C3d were detected in the lysed HEK293T cells (FIGS. 3B, 3C, and 3D) by ELISA, confirming the functionality of both the mRNA and the LNP delivery system. Moreover, C3d fusion did not affect the encapsulation of mRNA in LNP formulations as similar encapsulation efficiencies and nanoparticle sizes were observed for SP and SP-C3d as well as RBD and RBD-C3d (FIGS. 4A and 4B).

### Example 2. Immunogenicity Study of C3dfusion mRNA

For the vaccination study, a prime-and-boost vaccination schedule was employed. Female C57BL/6J mice were immunized intramuscularly with cKK-E12 LNPs encapsulating mRNA encoding SARS-CoV-2 antigens (SP, RBD) or C3d-fused SARS-CoV-2 antigens (mSP-C3d, mRBD-C3d) on day 0 (prime) and day 21 (boost). Three different doses of mRNA (0.01, 0.1, and 1 µg) were studied.

As a positive control, an adjuvanted cKK-E12 LNP formulation was prepared, in which 1% of the molar composition of PEG-lipid was replaced with lipopolysaccharide (LPS), a TLR-4 agonist (Oberli, M.A. et al. Lipid Nanoparticle Assisted mRNA Delivery for Potent Cancer Immunotherapy. Nano Lett 17, 1326-1335 (2017), the contents of which are incorporated herein by reference in their entirety). cKK-E12 LNPs containing firefly luciferase mRNA (mFFL) were also included as a negative control.

Following intramuscular (IM) injection of cKK-E12 LNPs encapsulating mFFL, robust expression of FFL was seen at the injection site in mice both 6 h and 24 h after injection, confirming the high transfection efficiency of LNPs (FIGS. 5A and 5B). Mouse sera were collected on day 14 and day 35 to evaluate antibody development using ELISA.

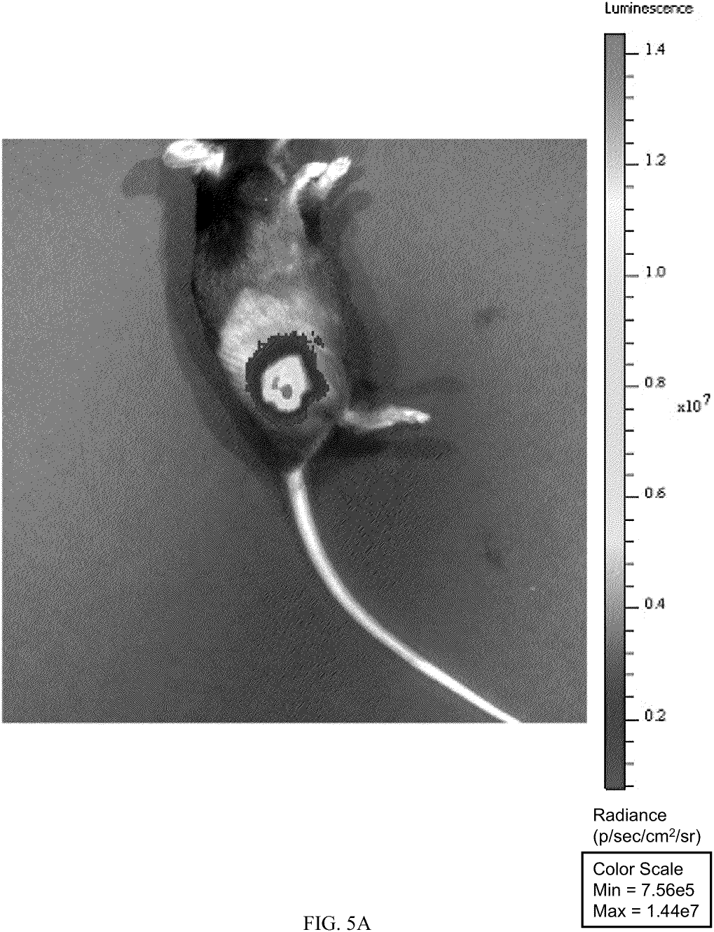

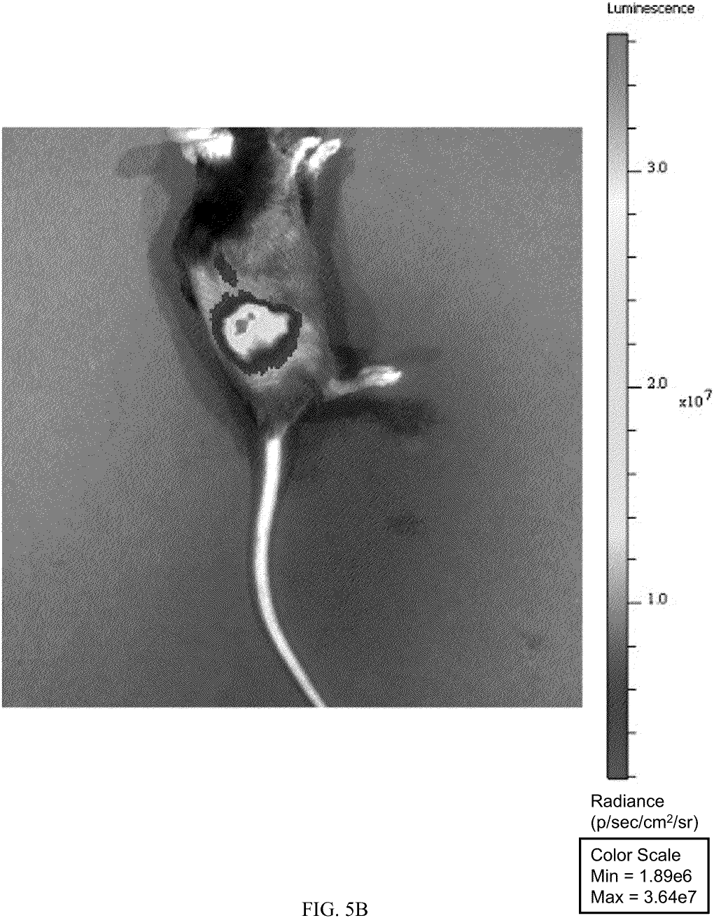

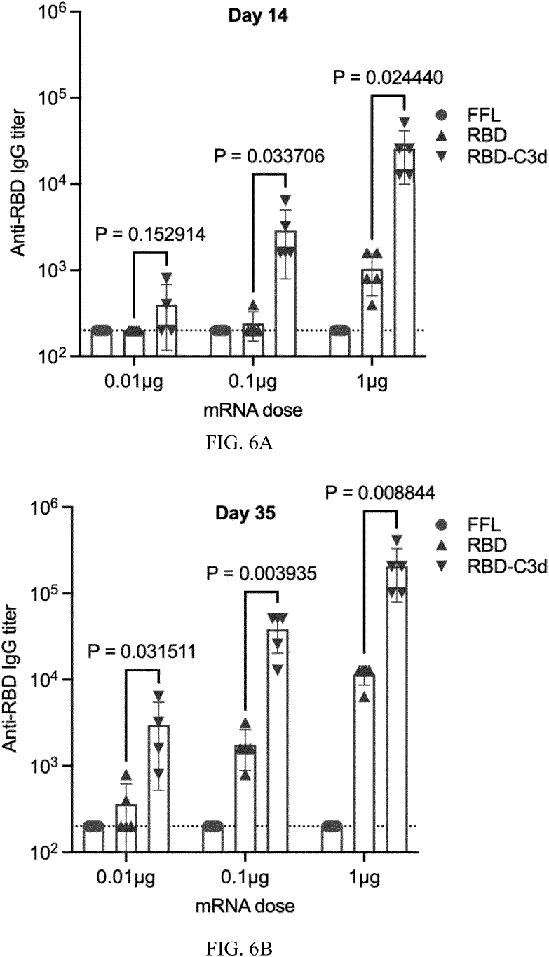

As shown in FIG. 1B, FIG. 1C, FIG. 6A, and FIG. 6B, dose-dependent titers of binding antibodies were observed in vaccinated mice after the prime and boost injection. Compared to mRNA vaccines encoding only SP or RBD (mSP, mRBD), both mSP-C3d and mRBD-C3d elicited a significantly higher level of IgG, demonstrating that the C3d fusion is a generally effective immunogenicity-enhancing strategy for mRNA vaccines.

Notably, the attachment of C3d mRNA to the mRNA encoding antigens could decrease the threshold dose for the specific antigen mRNA to elicit sufficient binding antibodies. For instance, the IgG titer induced by 1 µg mRNA encoding only SARS-CoV-2 antigens could be obtained by a 10-fold lower dose of mRNA (0.1 µg) encoding antigen fused with C3d.

Moreover, comparing mSP-C3d LNPs to mSP LNPs mixed with the traditional adjuvant LPS indicates that the C3d fusion is a more potent adjuvant technique than incorporating the conventional adjuvant, LPS, into the LNP formulation. Together, these results demonstrate that combining the C3d mRNA sequence with the sequence of mRNA encoding either the full-length spike protein or the receptor binding domain of SARS-CoV-2 into a single transcript enables the mRNA vaccine to induce a high level of antibodies in mice at a relatively low dose.

To compare the cellular responses induced by different mRNA vaccines, splenocytes from mice vaccinated with 0.1 µg mFFL, mSP, mSP-C3d and mSP/LPS, were collected and re-stimulated ex vivo with a library of SARS-CoV-2 peptides. The ELISPOT assay for IFN-γ secretion shows that ~250 spot forming units (SFU) per 2.5 × 10⁶ splenocytes was observed in the group treated by mSP-C3d, almost two-fold of that observed in the group treated by SP mRNA (FIG. 1D). Meanwhile, no significant difference in IFN-γ secretion between the groups immunized with mSP-C3d and mSP/LPS was observed.

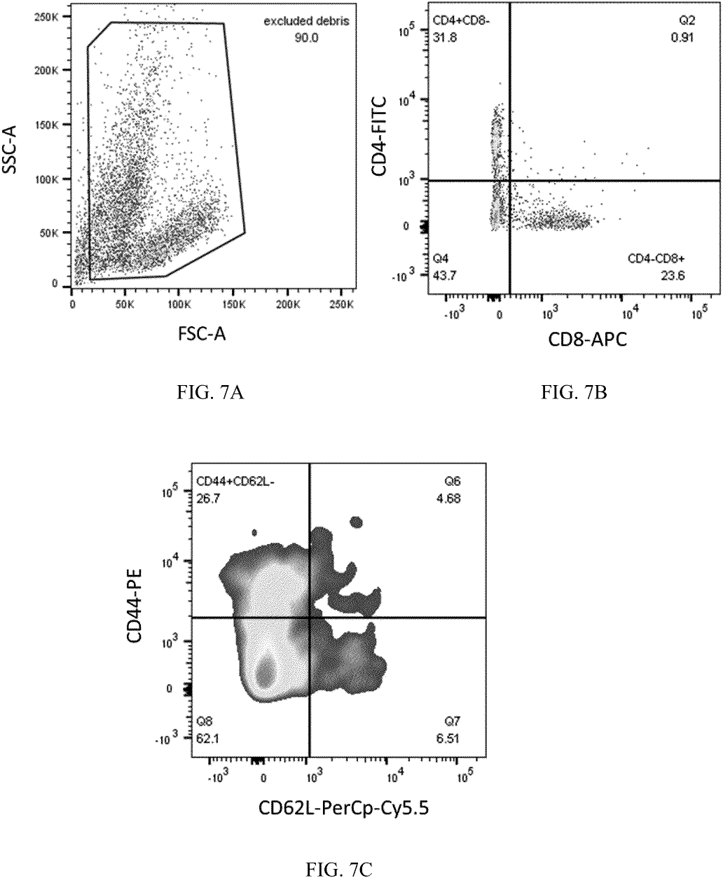

Additionally, flow cytometry results showed a significant increase in virus-specific CD4+ effector memory T (Tem) cells in splenocytes from mSP-vaccinated mice in comparison with mFFL-vaccinated mice (FIG. 1E, FIG. 7A, FIG. 7B, FIG. 7C) upon stimulation with peptide pools covering the SARS-CoV-2 SP. Notably, CD4+ Tem were higher in the group vaccinated with mSP-C3d and mSP/LPS than that in the group vaccinated with SP mRNA alone. C3d is proposed to function as a molecular adjuvant by efficiently targeting antigen to CD21/35 on B cells, which interacts with CD19 to regulate transmembrane signals during B cell activation. Next, the expression level of CD21 on B cells among mouse splenocytes was evaluated (FIG. 1F). A higher level of mean fluorescence intensity was detected in B cells from mice vaccinated with mSP-C3d than those from mice vaccinated with mSP or mSP/LPS. This suggests that the immunogenicity-enhancing property of C3d fusion mRNA is associated with its ability to mediate the interaction with CD21 receptors on B cells.

To directly assess antibody and immune responses in-vivo, at 6 h post-injection of LNPs, sera was collected for cytokine analysis by multiplex cytokine assay (FIG. 8A). Compared with mSP/LPS, which induced a pronounced release of pro-inflammatory cytokines such as TNF-α and IFN-γ in the mouse sera, the systemic cytokine levels triggered by mSP and mSP/C3d were much lower.

The immune-boosting effect of the C3d fusion strategy in wild-type COVID-19 mRNA vaccines motivated the assessment of whether this strategy could also enhance the immune response against the Delta variant of SARS-CoV-2 and motivated understanding whether the fusion of C3d to the antigen of interest is essential for achieving the observed immune potentiation or if similar increases in antibody titers could be achieved with the antigen and C3d separately expressed from independent mRNA transcripts.

To do this, four cohorts of C57BL/6J mice were respectively immunized with LNPs formulated with 1 µg of PBS, RBD_{Delta} mRNA, RBD_{Delta}-C3d fusion mRNA, or the mixture of RBD_{Delta} mRNA and free C3d (mC3d) mRNA. Expression of free, trimeric C3d was confirmed by Western blotting (FIG. 3D). The result shows that C3d fusion to RBD_{Delta} mRNA could also effectively enhance IgG antibodies against the RBD delta variant by at least one order of magnitude, from ~10⁴ to ~10⁵ (FIG. 1G). Moreover, this result also shows that free C3d mRNA mixed with RBD_{Delta} mRNA did not provide any boosting effect, implying that the adjuvant effect of C3d requires it to be directly fused with RBD_{Delta}.

Additionally, antibody levels across isotypes and subclasses against RBDs from variants of concern including WT, Alpha, Gamma, Beta, and Delta were measured to further characterize the antibodies raised in response to the Delta mRNA vaccines. Similar to the results observed for total anti-RBD_{Delta} IgG titers, vaccination with the C3d fusion vaccine led to the greatest humoral response and that vaccination with free C3d mRNA mixed with RBD_{Delta} mRNA did not yield a more significant response when compared to RBD_{Delta} mRNA vaccination alone (FIG. 1H). In particular, vaccination with mRBD_{Delta}-C3d resulted in significantly higher levels of anti-RBD_{Delta} IgG1, IgG2b and IgG2c when compared to vaccination with either mRBD_{Delta} or the combination of mRBD_{Delta} and mC3d (FIG. 8B). Analysis of the ratio of T_{H}1 associated antibodies (IgG2b and IgG2c) to T_{H}2 associated antibodies (IgG1) revealed a slight T_{H}2 shift for vaccination with mRBD_{Delta}-C3d when compared to vaccination with the other constructs (FIG. 8C).

### Example 3. Synergistic Effect of Immunostimlatory Lipids and C3d Fusion mRNA Following IM and IN Administration

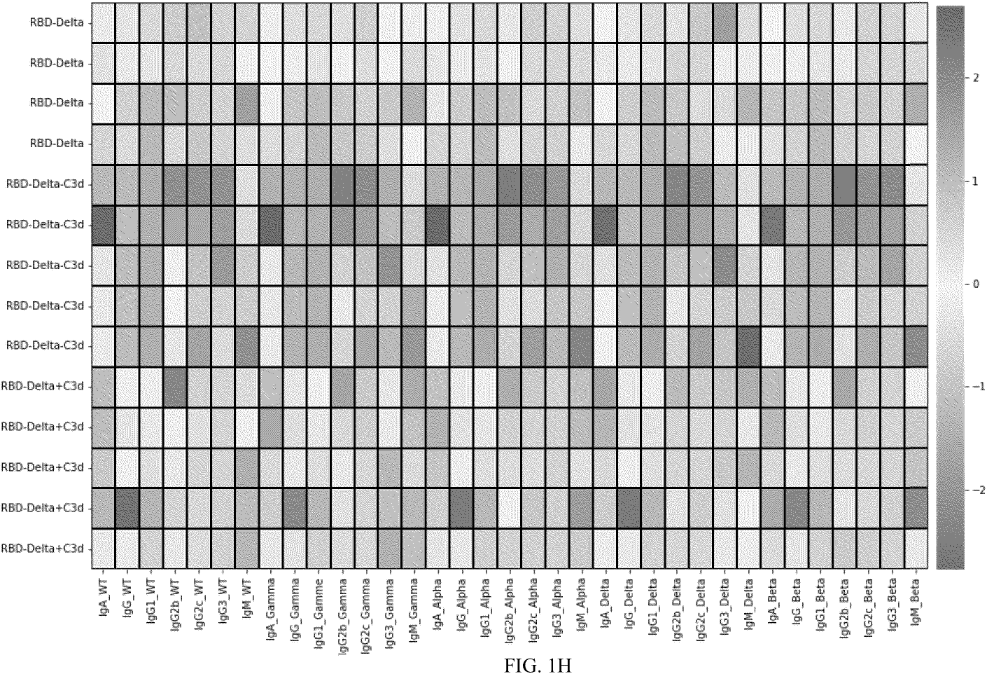

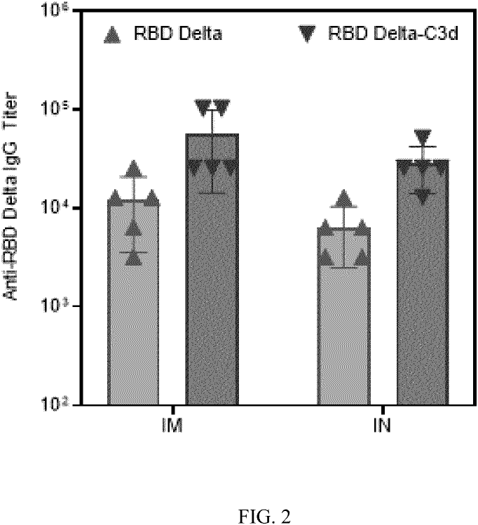

Subsequently, whether a self-adjuvating strategy could be employed to provide a more significant immunostimulatory effect was evaluated. Additionally, the impact of intranasal (IN) administration as a route for mRNA vaccine administration was examined in comparison to intramuscular (IM) administration. IN administration may allow for mRNA vaccines to be easily self-administered, significantly enhancing patient compliance. C57BL/6J mice were immunized with MC3 LNPs formulated with RBDdelta mRNA or RBDdelta-C3d fusion mRNA at the dosage of 1 µg mRNA following the same prime-boost vaccination schedule. ELISA assay of anti-mRBDdelta IgG in mouse sera shows no significant increase when contained within an MC3 LNP formulation for either route of administration. This observation confirms that the self-adjuvating effect of LNPs alone is not likely significant at low dosages (FIG. 2)

Moreover, IN administration of mRNA LNPs dosages were shown to be as effective as to the conventional IM injection in generating anti-RBD_{delta} IgG. A similar trend between each LNP sample-treated group was also observed after IN vaccination, demonstrating that the self-adjuvating strategy of an LNP formulation comprising a C3d fusion mRNA may be extended to other vaccination routes.

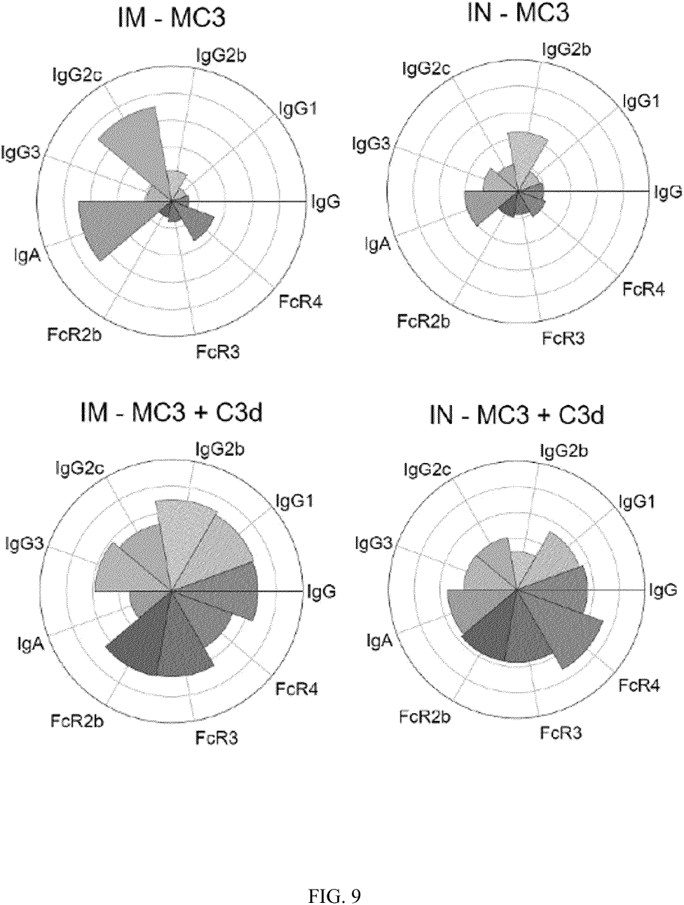

In addition to IgG, other antibody isotypes can drive immunity against SARS-CoV-2 in both mice and humans. Moreover, several studies have suggested that the ability of antibodies to drive clearance through binding to Fc receptors (FcRs) on the surface of innate immune cells is vital for the resolution of COVID-19. FcR-binding antibodies may be more resistant to mutations on variants of a concern than neutralizing antibodies. Therefore, the ability of these formulations to drive the induction of antibody subclasses and FcR-binding was measured. Polar plots of the mean percentile rank of the humoral response against RBD_{Delta} show that immunization without C3d generally resulted in a poor induction of antibody isotypes and FcRs against Delta RBD (FIG. 9).

### EXAMPLE EMBODIMENTS

Embodiment 1. A method of eliciting an enhanced immune response in a subject, the method comprising the step of administering to the subject a composition comprising:
a first polynucleotide sequence encoding an agent, and
a second polynucleotide sequence encoding a C3 complement protein degradation product (C3d) or a fragment thereof;
wherein the first polynucleotide sequence is operably connected to the second polynucleotide sequence;
and wherein the subject exhibits an enhanced immune response after administration of the composition.

Embodiment 2. The method of Embodiment 1, wherein the agent is an immunogen, a peptide, an antigen, an antibody, or a combination thereof.

Embodiment 3. The method of Embodiment 1 or 2, wherein the C3d comprises at least about 90% sequence identity to SEQ ID NO: 6, SEQ ID NO:8, SEQ ID NO:10, or SEQ ID NO:12.

Embodiment 4. The method of any one of Embodiments 1-3, wherein the second polynucleotide sequence comprises at least about 90% sequence identity to SEQ ID NO:9, SEQ ID NO:11, or a homolog thereof.

Embodiment 4. The method of any one of Embodiments 1-4, wherein the first polynucleotide sequence and the second polynucleotide sequence are operably connected through a linker.

Embodiment 6. The method of Embodiment 5, wherein the linker comprises a polynucleotide sequence selected from the group consisting of SEQ ID NO:13, SEQ ID NO:14, and SEQ ID NO:15.

Embodiment 7. The method of any one of Embodiments 1-6, wherein the second polynucleotide sequence comprises a C3d multimer selected from the group consisting of a C3d dimer, C3d trimer, C3d tetramer, and C3d pentamer.

Embodiment 8. The method of Embodiment 7, wherein the C3d multimer is a C3d trimer, wherein the C3d timer comprises at least about 80% sequence identity to SEQ ID NO:17 or SEQ ID NO:19.

Embodiment 9. The method of Embodiment 7 or 8, wherein the C3d multimer is a C3d trimer, wherein the C3d trimer is encoded by a sequence at least about 80% identical to SEQ ID NO:16, SEQ ID NO:18, or a homolog thereof.

Embodiment 10. The method of any one of Embodiments 1-9, wherein the subject is a human subject.

Embodiment 11. The method of Embodiment 10, wherein the subject is about 0-3 months, 0-6 months, 6-11 months, 12-15 months, 12-18 months, 19-23 months, 24 months, 1-2 years, 2-3 years, 4-6 years, 7-10 years, 11-12 years, 11-15 years, 16-18 years, 18-20 years, 20-25 years, 25-30 years, 30-35 years, 30-40 years, 35-40 years, 30-50 years, 30-60 years, 50-60 years, 60-70 years, 50-80 years, 70-80 years, 80-90 years, or older than 60 years.

Embodiment 12. The method of any one of Embodiments 1-9, wherein the subject is a domesticated animal.

Embodiment 13. The method of Embodiment 12, wherein the subject is a dog, cat, chicken, pig, cow, or horse.

Embodiment 14. The method of Embodiment any of Embodiments 1-13, wherein the first polynucleotide and the second polynucleotide are administered in a delivery vehicle comprising a nanocarrier selected from the group consisting of a lipid, a polymer and a lipo-polymeric hybrid.

Embodiment 15. The method of Embodiment any of Embodiments 1-13, wherein the first polynucleotide and the second polynucleotide are encapsulated in a lipid nanoparticle, polymer nanoparticle, virus-like particle, nanowire, exosome, or hybrid lipid/polymer nanoparticle.

Embodiment 16. The method of Embodiment 1 or 2, wherein the route of administration is intramuscular, intranodal, intravenous, intradermal, subcutaneous, intranasal, or epicardial.

Embodiment 17. The method of any one of Embodiments 1-16, wherein the composition is administered for treatment of an infectious disease.

Embodiment 18. A method for treating an infectious disease, comprising administering to a subject in need thereof a composition comprising:
a first polynucleotide encoding an agent, and
a second polynucleotide comprising a nucleic acid sequence encoding a C3 complement protein degradation product (C3d) or a fragment thereof;
wherein the first polynucleotide is operably connected to the second polynucleotide.

Embodiment 19. The method of Embodiment 17 or 18, wherein the infectious disease is a coronavirus, influenza virus, respiratory syncytial virus (RSFV), human immunodeficiency virus, zika virus, Epstein-Barr virus, herpes simplex virus, rabies, cytomegalovirus, mycobacterium tuberculosis, or a combination thereof.

Embodiment 20. The method of Embodiment 17 or 18, wherein the infectious disease is a SARS-CoV-2 or SARS-CoV-2-like virus.

Embodiment 21. The method of any one of Embodiments 17-20, wherein the agent is a spike protein (SP), a receptor binding domain (RBD), or a combination thereof.

Embodiment 22. The method of any one of Embodiments 17-20, wherein the agent is at least about 80% identical to SEQ ID NO:21.

Embodiment 23. The method of any one of Embodiments 17-20, wherein the agent is at least about 80% identical to SEQ ID NO:23 or SEQ ID NO:25.

Embodiment 24. The method of Embodiment 22, wherein the sequence encoding the agent is at least about 80% identical to SEQ ID NO:20 or a homology thereof.

Embodiment 25. The method of Embodiment 23, wherein the sequence encoding the agent is at least about 80% identical to SEQ ID NO:22, SEQ ID NO:24, or a homology thereof.

Embodiment 26. The method of any one of Embodiments 17-25, wherein the efficacy of treatment is determined by determining longevity of immunity, percent reduction in risk of disease cases in a population of subjects administered the composition, reduction of relative risk (RR) of disease among a population of subjects administered the composition, transmissibility, or a combination thereof.

Embodiment 27. The method of any one of Embodiments 17-25, wherein the subject administered the composition has decreased systemic cytokine expression compared to a subject administered a composition comprising the agent and lacking the second polynucleotide.

Embodiment 28. The method of any one of Embodiments 17-25, wherein the magnitude of antigen-antibody titers of the subject is higher than that of a subject administered a composition lacking the second polynucleotide.

Embodiment 29. The method of any one of Embodiments 17-25, wherein the Th1 immune response of the subject is higher than that of a subject administered a composition lacking the second polynucleotide.

Embodiment 30. The method of any one of Embodiments 1-16, wherein the subject is being treated for cancer.

Embodiment 31. A method for treating cancer, comprising administering to a subject in need thereof a composition comprising:
a first polynucleotide sequence encoding an agent, and
a second polynucleotide sequence encoding a C3 complement protein degradation product (C3d) or a fragment thereof;
wherein the first polynucleotide is operably connected to the second polynucleotide.

Embodiment 32. The method of any one of Embodiments 30-31, wherein the cancer is melanoma, colorectal cancer, high-risk melanoma, human papilloma virus, head and neck squamous carcinoma, non-small cell lung cancer, New York esophageal squamous cell carcinoma, or a combination thereof.

Embodiment 33. The method of Embodiment 32, wherein the agent is a HPV16-derived tumor antigen, E6 viral oncoprotein, E7 viral oncoprotein, melanoma-associated antigen, mucin1, or trophoblast glycoprotein.

Embodiment 34. A polynucleotide construct comprising:
a) a first polynucleotide sequence encoding an agent; and
b) a second polynucleotide encoding a C3 complement protein degradation product (C3d) or a fragment thereof;
wherein the first polynucleotide is operably connected to the second polynucleotide.

Embodiment 35. The construct of Embodiment 34, wherein the first polynucleotide and second polynucleotide are operably connected through a linker.

Embodiment 36. A messenger ribonucleic acid (mRNA) construct encoding an antigen and a C3 complement protein degradation product (C3d) or a fragment thereof.

Embodiment 37. A construct of Embodiment 36 which encodes multiple antigens, multiple copies of C3d or multiple copies of antigen and multiple copies of C3d.

Embodiment 38. A coding ribonucleic acid (RNA) sequence comprising RNA encoding an antigen and RNA encoding C3d.

Embodiment 39. A nanoparticle comprising the construct of any one of Embodiments 34-38.

Embodiment 40. A nanoparticle comprising at least two constructs of any one of Embodiments 34-38.

Embodiment 41. The construct of Embodiment 34, wherein the first polynucleotide, the second polynucleotide or both are circular mRNA.

Embodiment 42. A composition comprising the construct of Embodiment 34, wherein both the first polynucleotide and the second polynucleotide are encapsulated in a lipid nanoparticle.

Embodiment 43. The composition of Embodiment 42, wherein the lipid nanoparticle is ionizable.

Embodiment 44. The composition of Embodiment 42 or 43, further comprising a therapeutic agent.

Embodiment 35. The composition of any one of Embodiments 42-44 for use in any of the methods of Embodiments 1-33.

Embodiment 46. A method of making a composition comprising:

cloning a messenger ribonucleic acid (mRNA) encoding a C3 complement protein degradation product (C3d) or a fragment thereof into a cloning plasmid encoding an mRNA encoding an immunogen or antigen capable of inducing an immune response, to produce a C3d fusion mRNA.

Embodiment 47. The method of Embodiment 46, further comprising formulating the cloned RNA C3d fusion mRNA into a lipid nanoparticle.

Embodiment 48. The method of Embodiment 18 or 31, further comprising administering an additional therapeutic agent.

Embodiment 49. A method of eliciting an enhanced immune response in a cell, the method comprising contacting the cell with a composition comprising:
a first polynucleotide sequence encoding an agent, and
a second polynucleotide sequence encoding a C3 complement protein degradation product (C3d) or a fragment thereof;
wherein the first polynucleotide sequence is operably connected to the second polynucleotide sequence;
and wherein the cell exhibits an enhanced immune response after contact with the composition.

### References

1. Corbett, K.S. et al. SARS-CoV-2 mRNA vaccine design enabled by prototype pathogen preparedness. Nature 586, 567-571 (2020).

2. Polack, F.P. et al. Safety and Efficacy of the BNT162b2 mRNA Covid-19 Vaccine. New England Journal of Medicine 383, 2603-2615 (2020).

3. Baden, L.R. et al. Efficacy and Safety of the mRNA-1273 SARS-CoV-2 Vaccine. New England Journal of Medicine 384, 403-416 (2021).

4. Vogel, A.B. et al. BNT162b vaccines protect rhesus macaques from SARS-CoV-2. Nature 592, 283-289 (2021).

5. Barbier, A.J., Jiang, A.Y., Zhang, P., Wooster, R. & Anderson, D.G. The clinical progress of mRNA vaccines and immunotherapies. Nature Biotechnology (2022).

6. Pardi, N., Hogan, M.J., Porter, F.W. & Weissman, D. mRNA vaccines - a new era in vaccinology. Nature Reviews Drug Discovery 17, 261-279 (2018).

7. Buschmann, M.D. et al. Nanomaterial Delivery Systems for mRNA Vaccines. Vaccines 9 (2021).

8. Corbett, K.S. et al. SARS-CoV-2 mRNA vaccine design enabled by prototype pathogen preparedness. Nature 586, 567-571 (2020).

9. Kowalski, P.S., Rudra, A., Miao, L. & Anderson, D.G., Vol. 27 710-728 (Cell Press, 2019).

10. Patel, A.K. et al. Inhaled Nanoformulated mRNA Polyplexes for Protein Production in Lung Epithelium. Adv Mater 31, e1805116 (2019).

11. Zhang, N.N. et al. A Thermostable mRNA Vaccine against COVID-19. Cell 182, 1271-1283 e1216 (2020).

12. McKay, P.F. et al. Self-amplifying RNA SARS-CoV-2 lipid nanoparticle vaccine candidate induces high neutralizing antibody titers in mice. Nat Commun 11, 3523 (2020).

13. Pulendran, B., S. Arunachalam, P. & O’Hagan, D.T. Emerging concepts in the science of vaccine adjuvants. Nature Reviews Drug Discovery 20, 454-475 (2021).

14. Alameh, M.-G. et al. Lipid nanoparticles enhance the efficacy of mRNA and protein subunit vaccines by inducing robust T follicular helper cell and humoral responses. Immunity 54, 2877-2892.e2877 (2021).

15. Tahtinen, S. et al. IL-1 and IL-1ra are key regulators of the inflammatory response to RNA vaccines. Nature Immunology 23, 532-542 (2022).

16. Swaminathan, G. et al. A Tetravalent Sub-unit Dengue Vaccine Formulated with Ionizable Cationic Lipid Nanoparticle induces Significant Immune Responses in Rodents and Non-Human Primates. Scientific Reports 6 (2016).

17. Miao, L. et al. Delivery of mRNA vaccines with heterocyclic lipids increases anti-tumor efficacy by STING-mediated immune cell activation. Nature Biotechnology 37, 1174-1185 (2019).

18. Ahn, J. & Barber, G.N. STING signaling and host defense against microbial infection. Experimental & Molecular Medicine 51, 1-10 (2019).

19. Hassett, K.J. et al. Optimization of Lipid Nanoparticles for Intramuscular Administration of mRNA Vaccines. Molecular Therapy - Nucleic Acids 15, 1-11 (2019).

20. Sabnis, S. et al. A Novel Amino Lipid Series for mRNA Delivery: Improved Endosomal Escape and Sustained Pharmacology and Safety in Non-human Primates. Molecular Therapy, 1509-1519 (2018).

21. Maier, M.A. et al. Biodegradable Lipids Enabling Rapidly Eliminated Lipid Nanoparticles for Systemic Delivery of RNAi Therapeutics. Molecular Therapy 21, 1570-1578 (2013).

22. Toapanta, F.R. & Ross, T.M. Complement-Mediated Activation of the Adaptive Immune Responses: Role of C3d in Linking the Innate and Adaptive Immunity. Immunologic Research 36, 197-210 (2006).

23. Bower, J.F. & Ross, T.M. in Current Topics in Complement 249-264 (2006).

24. Rickert, R.C. Regulation of B lymphocyte activation by complement C3 and the B cell coreceptor complex. Current Opinion in Immunology 17, 237-243 (2005).

25. Wang, Z. et al. Enhanced SARS-CoV-2 neutralization by dimeric IgA. Science Translational Medicine (2021).

26. Sterlin, D. et al. IgA dominates the early neutralizing antibody response to SARS-CoV-2. Science Translational Medicine 13 (2021).

27. Boyaka, P.N. Inducing Mucosal IgA: A Challenge for Vaccine Adjuvants and Delivery Systems. The Journal of Immunology 199, 9-16 (2017).

28. Allie, S.R. et al. The establishment of resident memory B cells in the lung requires local antigen encounter. Nature Immunology 20, 97-108 (2018).

29. Morabito, K.M. et al. Memory Inflation Drives Tissue-Resident Memory CD8+ T Cell Maintenance in the Lung After Intranasal Vaccination With Murine Cytomegalovirus. Frontiers in Immunology 9 (2018).

30. Phua, K.K.L., Staats, H.F., Leong, K.W. & Nair, S.K. Intranasal mRNA nanoparticle vaccination induces prophylactic and therapeutic anti-tumor immunity. Scientific Reports 4 (2014).

31. Mai, Y. et al. Intranasal delivery of cationic liposome-protamine complex mRNA vaccine elicits effective anti-tumor immunity. Cellular Immunology 354 (2020).

32. Tarke, A. et al. Impact of SARS-CoV-2 variants on the total CD4(+) and CD8(+) T cell reactivity in infected or vaccinated individuals. Cell Rep Med 2, 100355 (2021).

33. Schulien, I. et al. Characterization of pre-existing and induced SARS-CoV-2-specific CD8(+) T cells. Nat Med 27, 78-85 (2021).

34. Le Bert, N. et al. SARS-CoV-2-specific T cell immunity in cases of COVID-19 and SARS, and uninfected controls. Nature 584, 457-462 (2020).

35. Grifoni, A. et al. Targets of T Cell Responses to SARS-CoV-2 Coronavirus in Humans with COVID-19 Disease and Unexposed Individuals. Cell 181, 1489-1501 e1415 (2020).

36. Peng, Y. et al. Broad and strong memory CD4(+) and CD8(+) T cells induced by SARS-CoV-2 in UK convalescent individuals following COVID-19. Nat Immunol 21, 1336-1345 (2020).

37. Oberhardt, V. et al. Rapid and stable mobilization of CD8+ T cells by SARS-CoV-2 mRNA vaccine. Nature 597, 268-273 (2021).

38. Reinscheid, M. et al. COVID-19 mRNA booster vaccine induces transient CD8+ T effector cell responses while conserving the memory pool for subsequent reactivation. Nature Communications 13 (2022).

39. Liu, J. et al. CD8 T cells contribute to vaccine protection against SARS-CoV-2 in macaques. Science Immunology 7 (2022).

40. Kauffman, K.J. et al. Optimization of Lipid Nanoparticle Formulations for mRNA Delivery in Vivo with Fractional Factorial and Definitive Screening Designs. Nano Lett 15, 7300-7306 (2015).

41. Oberli, M.A. et al. Lipid Nanoparticle Assisted mRNA Delivery for Potent Cancer Immunotherapy. Nano Lett 17, 1326-1335 (2017).

42. Alu, A. et al. Intranasal COVID-19 vaccines: From bench to bed. eBioMedicine 76 (2022).

43. Lu, L.L., Suscovich, T.J., Fortune, S.M. & Alter, G. Beyond binding: antibody effector functions in infectious diseases. Nature Reviews Immunology 18, 46-61 (2017).

44. Zohar, T. et al. Compromised Humoral Functional Evolution Tracks with SARS-CoV-2 Mortality. Cell 183, 1508-1519.e1512 (2020).

45. Kaplonek, P. et al. Early cross-coronavirus reactive signatures of humoral immunity against COVID-19. Science Immunology 6 (2021).

46. Kaplonek, P. et al. mRNA-1273 vaccine-induced antibodies maintain Fc effector functions across SARS-CoV-2 variants of concern. Immunity 55, 355-365.e354 (2022).

47. Harrod, K. et al. The Fc-mediated effector functions of a potent SARS-CoV-2 neutralizing antibody, SC31, isolated from an early convalescent COVID-19 patient, are essential for the optimal therapeutic efficacy of the antibody. Plos One 16 (2021).

48. Rosenblum, H.G. et al. Safety of mRNA vaccines administered during the initial 6 months of the US COVID-19 vaccination programme: an observational study of reports to the Vaccine Adverse Event Reporting System and v-safe. The Lancet Infectious Diseases (2022).

49. Dempsey, P.W., Allison, M.E.D., Akkaraju, S., Goodnow, C.C. & Fearon, D.T. C3d of complement as a molecular adjuvant: Bridging innate and acquired immunity. Science 271, 348-350 (1996).

50. Ross, T.M., Xu, Y., Bright, R.A. & Robinson, H.L. C3d enhancement of antibodies to hemagglutinin accelerates protection against influenza virus challenge. Nature Immunology 1, 127-131 (2000).

51. Bower, J., Sanders, K. & Ross, T. C3d Enhances Immune Responses Using Low Doses of DNA Expressing the HIV-1 Envelope from Codon-Optimized Gene Sequences. Current HIV Research 3, 191-198 (2005).

52. Green, T.D. et al. C3d enhancement of neutralizing antibodies to measles hemagglutinin. Vaccine 20, 242-248 (2001).

53. He, Y.G. et al. A novel C3d-containing oligomeric vaccine provides insight into the viability of testing human C3d-based vaccines in mice. Immunobiology 223, 125-134 (2018).

54. Zhang, D. et al. Construction and immunogenicity of DNA vaccines encoding fusion protein of murine complement C3d-p28 and GP5 gene of porcine reproductive and respiratory syndrome virus. Vaccine 29, 629-635 (2011).

55. Green, T.D., Montefiori, D.C. & Ross, T.M. Enhancement of antibodies to the human immunodeficiency virus type 1 envelope by using the molecular adjuvant C3d. J Virol 77, 2046-2055 (2003).

56. Watanabe, I. et al. Protection against influenza virus infection by intranasal administration of C3d-fused hemagglutinin. Vaccine 21, 4532-4538 (2003).

57. Yang, S. et al. Fusion of C3d molecule with neutralization epitope(s) of hepatitis E virus enhances antibody avidity maturation and neutralizing activity following DNA immunization. Virus Res 151, 162-169 (2010).

58. Wang, L., Sunyer, J.O. & Bello, L.J. Fusion to C3d enhances the immunogenicity of the E2 glycoprotein of type 2 bovine viral diarrhea virus. J Virol 78, 1616-1622 (2004).

59. Zhang, Z. et al. Fusion to chicken C3d enhances the immunogenicity of the M2 protein of avian influenza virus. Virol J7, 89 (2010).

60. Chen, G. et al. Clinical and immunological features of severe and moderate coronavirus disease 2019. Journal of Clinical Investigation 130, 2620-2629 (2020).

61. Levin, E.G. et al. Waning Immune Humoral Response to BNT162b2 Covid-19 Vaccine over 6 Months. New England Journal of Medicine 385 (2021).

62. Goldberg, Y. et al. Waning Immunity after the BNT162b2 Vaccine in Israel. New England Journal of Medicine 385 (2021).

63. Ndeupen, S. et al. The mRNA-LNP platform’s lipid nanoparticle component used in preclinical vaccine studies is highly inflammatory. iScience 24 (2021).

64. Boudreau, C.M. & Alter, G. Extra-Neutralizing FcR-Mediated Antibody Functions for a Universal Influenza Vaccine. Frontiers in Immunology 10 (2019).

65. Meyer, M. et al. Ebola vaccine-induced protection in nonhuman primates correlates with antibody specificity and Fc-mediated effects. Science Translational Medicine 13 (2021).

66. Neidich, S.D. et al. Antibody Fc effector functions and IgG3 associate with decreased HIV-1 risk. Journal of Clinical Investigation 129, 4838-4849 (2019).

67. Zohar, T. et al. Upper and lower respiratory tract correlates of protection against respiratory syncytial virus following vaccination of nonhuman primates. Cell Host & Microbe 30, 41-52.e45 (2022).

68. Yamin, R. et al. Fc-engineered antibody therapeutics with improved anti-SARS-CoV-2 efficacy. Nature 599, 465-470 (2021).

69. Heyes, J., Palmer, L., Bremner, K. & MacLachlan, I. Cationic lipid saturation influences intracellular delivery of encapsulated nucleic acids. Journal of Controlled Release 107, 276-287 (2005).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A messenger ribonucleic acid (mRNA) construct comprising:
a) a first mRNA sequence encoding an agent; and
b) a second mRNA encoding a C3 complement protein degradation product (C3d) or a fragment thereof; wherein the first mRNA is operably connected to the second mRNA.

2. The construct of claim 1, wherein the first RNA and second RNA are operably connected through a linker.

3. A messenger ribonucleic acid (mRNA) construct encoding an antigen and a C3 complement protein degradation product (C3d) or a fragment thereof.

4. A construct of claim 3 which encodes multiple antigens, multiple copies of C3d or multiple copies of antigen and multiple copies of C3d.

5. A coding ribonucleic acid (RNA) sequence comprising RNA encoding an antigen and RNA encoding C3d.

6. A nanoparticle comprising the construct of claim 1.

7. A nanoparticle comprising at least two constructs of claim 1.

8. The construct of claim 7, wherein the first polynucleotide, the second polynucleotide or both polynucleotides are circular mRNA.

9. A composition comprising the construct of claim 8, wherein both the first polynucleotide and the second polynucleotide are encapsulated in a nanoparticle.

10. The composition of claim 9, wherein the nanoparticle is ionizable.

11. The composition of claim 9, further comprising a therapeutic agent.

12. A method of inducing a response to an antigen in a cell, the method comprising contacting the cell with a composition comprising:
a first polynucleotide sequence encoding an agent, and
a second polynucleotide sequence encoding a C3 complement protein degradation product (C3d) or a fragment thereof;
wherein the first polynucleotide sequence is operably connected to the second polynucleotide sequence; and wherein the response is induced after contact with the composition.

13. A method of making a composition comprising:
cloning a messenger ribonucleic acid (mRNA) encoding a C3 complement protein degradation product (C3d) or a fragment thereof into a cloning plasmid encoding an mRNA encoding an immunogen or antigen capable of inducing an immune response, to produce a C3d fusion mRNA.

14. The method of claim 13, further comprising formulating the cloned RNA C3d fusion mRNA into a nanoparticle.

15. The method of claim 13, further comprising administering an additional therapeutic agent.

16. The construct of claim 3, wherein the RNA is chemically modified RNA.
